(19) United States
(12) Patent Application Publication
(10) Pub. No.: US 2013/0309757 A1
(43) Pub. Date: Nov. 21, 2013

(54) METHOD AND APPARATUS FOR PRODUCING CELLS AND FAT SOLUBLE MATERIALS BY CELL CULTURE

(71) Applicant: Sung-Chun Kim, Seoul (KR)

(72) Inventor: Sung-Chun Kim, Seoul (KR)

(21) Appl. No.: 13/995,068

(22) PCT Filed: Dec. 16, 2011
(86) PCT No.: PCT/KR11/09716

§ 371 Date: Aug. 6, 2013

(30) Foreign Application Priority Data
Dec. 17, 2010 (KR) 10-2010-0129852
Jul. 26, 2011 (KR) 10-2011-0074014

(51) Int. Cl. C12N 1/12

(52) U.S. Cl. CPC C12N 1/12
USPC 435/257.3; 435/294.1

(57) ABSTRACT

The present invention relates to a method and apparatus for producing cells without injury and fat-soluble materials by from cell culturing in an inexpensive and highly efficient manner. The apparatus according to the present invention comprises a culturing device 10, a solvent device 20, a mixing device 30, a separation device 40, a fractionation device 50, a cell accommodation device 60, and a fat-soluble material solvent accommodation device 70.

### TECHNICAL FIELD

The present invention relates to a method and apparatus for producing cells and fat-soluble materials by cell culturing.

More particularly, the present invention relates a method and apparatus for producing cells without injury and fat-soluble materials by from cell culturing in an inexpensive and highly efficient manner.

### BACKGROUND ART

Biomass obtained by culturing cells, such as microalgae, has been used as a raw material for health functional foods and medicines, with the utility thereof extending to the production of feedstuffs, alternative energy raw materials and biochemical materials, etc.

When cultured in bioreactors, cells or microorganisms are excellent producers of fat-soluble materials. Upon an ultra-high density culture (particularly, on an industrial scale, e.g., exceeding about 100 g/L biomass), a growth inhibitor may be secreted from the cells, with a sequent reduction in the productivity of biomass and the content and productivity of fat-soluble materials.

Permeability of solvents for extracting fat-soluble materials into cells or microorganisms differs from one species or strain to another. Thus, the efficiency at which fat-soluble materials can be extracted using a fat-soluble material extracting solvent may vary depending on species or strains of the source.

The economics of mass production of biomass is secured by scale-up of cell culture. For batch fermentation, for example, its economics depends on the size of a stirred tank or an airlift fermenter.

Continuous perfusion is a type of cell culture in which cells are immobilized in a part of a system, and nutrients/oxygen are allowed to flow through the stationary cells, thus obtaining a medium which is free of cells but contains a product of interest. By continuous perfusion, cells can be cultured for weeks or months in a fermenter while a fresh medium is continuously fed to the fermenter. This technique enables cells to be cultured at a high density, with the concomitant conversion of substrates into products at high efficiency, and guarantees the effective use of the fermenter. Therefore, when continuous perfusion is used, the goal of cell culture can be achieved even in a small-size fermenter.

In continuous perfusion, cells are separated from the cell culture solution containing the product of interest and are directed toward a fermenter while the remainder is harvested. In this regard, the cell separator must be sufficiently elaborate because cells are very sensitive to physical impacts. In addition, the cell separation apparatus is required to be operated aseptically without processional problems. Moreover, the cell separator must be simple and robust, extendable to an economic scale, sanitary, and have sufficient sealability to grow dangerous organisms.

Continuous perfusion makes use of an ultrasonic resonance field, a gravitational settling device, a spin filter device, a filtration membrane, or a centrifuge in stagnating or separating cells.

A cell separator employing an ultrasonic resonance field, a gravitational settling device, or a filtration membrane is able to perform the function of cell separation in an almost permanent manner using even a simple device at very low power. The cell separator enjoys the greatest advantage of, among others, separating cells while maintaining the cells intact without the help of an additional mechanical device.

Centrifugation, a conventional technique of harvesting cells, is difficult to apply to an on-line system, whereas an ultrasonic resonance field, gravitational settling, or a filtration membrane is applicable. In addition, the application of an ultrasonic resonance field, gravitational settling, or a filtration membrane to a cell separator allows the fermenter to perform on-line clarification and perfusion culture. Therefore, an ultrasonic resonance field device, a gravitational settling device, or a filtration membrane device, when applied to the recovery of cells or extracellular products, is expected to be an alternative to the conventional process.

Because it is conducted at very low power, separation of cells or microorganisms by an ultrasonic resonance field, gravitational settling or a filtration membrane has almost no influence on the life and death of microorganisms or cells, and is used to maintain cells without provoking cell death in a perfusion culture method for culturing plant cells, insect cells or animal cells at a high density. Further, it is applied to continuous perfusion culture for producing monoclonal antibodies.

Cells contain various materials including secondary metabolites such as physiologically active materials as well as primary metabolites such as lipids, proteins and carbohydrates. Proteins are most abundantly found in cells, but can be obtained, for the most part, using cells themselves. In view of use of materials produced in cells not cells themselves, attention may be paid to lipids, carbohydrates, pigments, vitamins, minerals and other special compounds.

In addition, intermediates found in the synthesis and degradation of carbohydrates, proteins, nucleic acids, and lipids, and materials involved in the regulation of such metabolisms, are necessary for cells, and thus can be obtained from cells.

Cellular biomass can be converted into bioenergy by a biorefinery which integrates thermochemical and biochemical techniques to produce liquid fuels such as bioethanol, biobutanol, biodiesel, etc., and gas fuels such as hydrogen, methane, etc. depending on process. Representative biofuels are biodiesel and bioethanol that can substitute for diesel and gasoline, respectively.

Although not indispensible for the growth of plants, useful compounds (predominantly, secondary metabolites) exist in various forms and large amounts. As many as about 100,000 compounds have been identified thus far. In many case, specific individual compounds are detected only in several species or families of plants. Representative among the useful metabolites are alkaloids, phenolic compounds, and terpenes, etc.

In conventional methods for extracting valuable materials from cellular biomass, cells are dehydrated as much as possible by centrifugation, filtration and drying processes, all inhibitory of the growth of cells, and undergo a cell disruption process to separate and purify valuable materials.

However, these conventional extraction methods make it difficult to reculture or reuse cells because the cells are disrupted during the dehydration and extraction processes, and are disadvantageous in that they are operated at a high cost upon mass culture because of such complicated processes.

Microalgae can be cultured in an autotrophic manner which is based on photosynthesis utilizing light energy, carbon dioxide, and water, in a heterotrophic manner in which they cannot utilize light as an energy source, but obtain carbon by feeding on organic material present in other organisms, or in a mixotrophic manner which may be either autotrophic or heterotrophic at different times in their life.

Fermentation of organic waste generates biogas and organic wastewater. When cultured in organic wastewater, microalgae grow by absorbing organic materials, and nitrogen and phosphorous components. Thus, microalgae are expected to remove organic materials, and nitrogen and phosphorous components, with the concomitant production of biomass useful as a raw material for bioenergy and biochemicals, and as biologically active materials and fish feed. In addition, because the cultured microalgae are rich in useful protein, they are expected to be used as high-protein feedstuffs for animals. Therefore, a combination of an organic waste fermentation system and a microalgae culture system could produce biogas and biomass on a mass scale in addition to clarifying organic wastewater.

### DISCLOSURE

### Technical Problem

It is an object of the present invention to provide a method and apparatus for producing intact cells and fat-soluble materials from a cell culture solution at a low cost and high efficiency.

It is another object of the present invention to provide a method and apparatus for producing cells and fat-soluble materials by cell culture solution, in which cultured cells are fermented to increase the content of fat-soluble materials, and modified such that a fat-soluble extracting solvent can readily penetrate into the cells and dissolve intracellular fat-soluble materials, thereby improving the production efficiency of intact cells and fat-soluble materials.

It is a further object of the present invention to provide a method and apparatus for producing cells and fat-soluble materials at high efficiency by concentrating and/or fermenting cultured cells.

### Technical Solution

In accordance with an aspect thereof, the present invention provides a method and an apparatus for producing cells and fat-soluble materials by cell culture.

The method according to the present invention comprises: culturing cells containing fat-soluble materials; contacting and mixing the cell culture solution with a fat-soluble material extracting solvent to exude the fat-soluble material into the fat-soluble material extracting solvent; separating the cells from the mixed solution; fractionating the remaining solution free of cells into a fat-soluble material-solvent layer containing the fat-soluble material, and a water layer.

Preferably, the method according to the present invention further comprises concentrating cells of the cell culture solution.

Preferably, the method according to the present invention further comprises maturing cells of the cell culture solution to allow the cells to have an increased content of the fat-soluble material, and modifying the cells such that the fat-soluble material extracting solvent readily penetrates into the cells to dissolve the fat-soluble material easily.

In a preferred embodiment of the method, the cells separated from the mixed solution are re-cultured, the resulting cell culture solution are mixed and contacted with the fat-soluble material extracting solvent or the fractionated fat-soluble material-solvent to further dissolve the fat-soluble material of the cells in the fat-soluble material extracting solvent. The subsequent steps are repeated in such a way that a serial process of re-culturing the cells, re-dissolving the fat-soluble material, re-separating the cells, and re-fractionating the fat-soluble material-solvent is repeated 2 or more times, whereby the cells can be grown to a high density of interest, with concomitant production of the fat-soluble material-solvent containing a desired concentration of the fat-soluble material.

The method according to the present invention comprises: culturing cells containing fat-soluble materials; concentrating the cells of the cell culture solution; maturing the cells to allow the cells to have an increased content of the fat-soluble material, and modifying the cells such that a fat-soluble material extracting solvent readily penetrates into the cells to dissolve the fat-soluble material easily; contacting and mixing the concentrated and matured cell culture solution with the fat-soluble material extracting solvent to exude the fat-soluble material into the fat-soluble material extracting solvent; repeating a series of processes of dividing a fat-soluble material-solvent as a separate layer in the fat-soluble material extracting solvent, said fat-soluble material-solvent containing the fat-soluble material, contacting the separate fat-soluble material-solvent with the matured cell culture solution to again divide the fat-soluble material-solvent as a separate layer in the fat-soluble material extracting solvent; and obtaining the cells, and the fat-soluble material-solvent in which the fat-soluble material is dissolved, separately.

Preferably, in the method according to the present invention, while the cells are cultured, the cells ferment organic waste to produce organic wastewater containing low-molecular weight organic acids and biogas, said organic wastewater being recycled as nutrients in culturing the cells.

In the method, preferably, the organic wastewater is diluted to an extent of a TCOD (total chemical oxygen demand) of 100 to 10,000 mg/l and a nitrogen concentration of 100 to 800 mg/l before being used in culturing the cells.

In the method, preferably, the cells are cultured in organic wastewater containing an organic material, nitrogen and phosphorus, so that the organic material, nitrogen, and phosphorous are removed from the organic wastewater by using as nutrients of the cells, thereby purifying the organic wastewater.

In the method, preferably, the fat-soluble material extracting solvent is a hydrocarbon solvent.

Preferably, the method further comprises recovering a cell growth inhibitor and/or a pathogen from the cell culture solution, said cell growth inhibitor and said pathogen being secreted from the cells.

In the method, preferably, the cells are separated from the mixed solution by applying a gravitational settling process or an ultrasonic resonance field to the mixed solution.

In the method, preferably, the cell is mixed with the fat-soluble material extracting solvent through either or both of vibrational disintegration and stirring, whereby the cell culture solution is brought into improved contact with the fat-soluble material extracting solvent.

In the method, preferably, the cells from which the fat-soluble material is extracted are used to produce at least one compound selected from among cellulose, hemicelluloses, monosaccharides, and oligosaccharides.

In the method, preferably, hydrogen is generated and obtained when the compound is produced.

In the method, preferably, the fractionated fat-soluble material-solvent is used as a source from which a fatty acid or a pigment is separated.

In the method, preferably, the cells are concentrated by applying an ultrasonic resonance field or a gravitational settling method to the cell culture solution.

In the method, preferably, the cell culture solution is supplemented with a carbon source absolutely or almost free of a limiting nutrient to establish a nutrient limiting condition which induces the cells to produce the fat-soluble material.

In the method, preferably, the limiting nutrient is selected from the group consisting of a nitrogen source, a carbon source, a phosphate source, a vitamin source, a trace metal source, a major metal source, a silica source, and a combination thereof.

In the method, preferably, the cell culture is controlled in dissolved oxygen content so as to induce the cells to mature.

In accordance with an aspect thereof, the present invention provides an apparatus for producing cells and fat-soluble materials by cell culture.

The apparatus according to the present invention comprises: a culturing device for culturing cells containing fat-soluble materials; a solvent device for storing and providing a fat-soluble material extracting solvent capable of dissolving the fat-soluble material in the cells; a mixing device for mixing the cell culture solution transferred from the culturing device with the fat-soluble material extracting solvent transferred from the solvent device; a separation device for separating the cells from the mixed solution of the mixing device; a fractionation device for fractionating the cell-removed solution of the separation device into a fat-soluble material-solvent layer comprising the fat-soluble material extracting solvent and the fat-soluble material, and a water layer; a cell accommodation device for accommodating or treating the cells separated in the separation device; and a fat-soluble material solvent accommodation device for accommodating or treating the fat-soluble material-solvent layer divided in the fractionation device.

In the apparatus, preferably, the separation device comprises an ultrasonic resonance field generator for applying an ultrasonic resonance field to the mixed solution to separate the cells from the mixed solution, or a gravitational settling device for applying gravitational settling to the mixed solution to separate the cells from the mixed solution.

Preferably, the apparatus according to the present invention further comprises a cell circulation line through which the cells separated in the separation device is recycled to the culturing device; and a solvent circulation line through which the fat-soluble material-solvent fractioned in the fractionation device is recycled to the solvent device.

Wherein the cell accommodation device functions to accommodate or treat a high density of the cells finally separated in the separation device after said cells are recycled once or more times through cell circulation line to the culturing device and re-cultured; and the fat-soluble material-solvent accommodation device functions to accommodate or treat the fat-soluble material-solvent finally fractionated in the fractionation device after the fat-soluble material-solvent fraction is recycled once or more times through the solvent circulation line to the fraction device and re-fractionated.

Preferably, the apparatus according to the present invention further comprises: a first peristaltic pump for feeding the cell culture solution of the culturing device and the fat-soluble material extracting solvent of the solvent device in a predetermine amount to the mixing device; a second peristaltic pump for selectively delivering the cells separated in the separation device to the culturing device through the cell circulation line or to the cell accommodation device according to the density of the cells; and a third peristaltic pump for transferring the fat-soluble material-solvent fractionated in the fractionation device to the solvent device or the fat-soluble material solvent accommodation device according to the concentration of the fat-soluble material-solvent.

In the apparatus, preferably, the mixing device further comprises a device selected from among a vibrational disintegrator, a stirrer, and a combination thereof. Said vibrational disintegrator functions to disassemble aggregates of cells and said stirrer functions to the mixed solution, whereby the cell culture solution can brought into improved contact with the fat-soluble material extracting solvent.

Preferably, the apparatus further comprises a fermenter for fermenting organic waste to produce organic wastewater and biogas; a biogas accommodation device for capturing the biogas; and an organic wastewater accommodation device for storing the organic wastewater. Wherein the organic wastewater fed from the organic wastewater accommodation device is transferred to the culturing device and used in culturing the cells.

An apparatus according to the present invention comprises, a culturing device for culturing cells containing fat-soluble materials; a solvent device for storing and providing a fat-soluble material extracting solvent capable of dissolving the fat-soluble material of the cells; a cell concentration device for concentrating cells of the cell culture solution from the culturing device; a cell maturation device for maturing the concentrated cells of the cell culture solution; a mixing device for mixing the cell culture solution transferred from the culturing device with the fat-soluble material extracting solvent transferred from the solvent device 20; a separation device for separating the cells from the mixed solution of the mixing device; a fractionation device for fractionating the cell-removed solution of the separation device into a fat-soluble material-solvent layer comprising the fat-soluble material extracting solvent and the fat-soluble material, and a water layer; a cell accommodation device for accommodating or treating the cells separated in the separation device; a fat-soluble material solvent accommodation device for accommodating or treating the fat-soluble material-solvent layer divided in the fractionation device; and a water accommodation device for accommodating or treating the water separated in the cell concentration device and the fractionation device.

In the apparatus, preferably, the cell maturation device, the mixing device, and the separation device are assembled into an integrated separation device.

In the apparatus, preferably, the integrated separation device is designed to allow the fat-soluble material extracting solvent to be fed to a lower portion of the cell culture solution, and comprises an inlet line through which the fat-soluble material extracting solvent is introduced from the solvent device, a spout tube, connected to the end of the inlet line, for spouting the fat-soluble material extracting solvent upward from the bottom of the cell culture solution, a recovery line through the fat-soluble material extracting solvent of the integrated separation device plus the fat-soluble material dissolved in the fat-soluble material extracting solvent are recovered to the solvent device, and a peristaltic pump, located on both the inlet line and the recovery line, for functioning to provide a pressure necessary for transferring the solvents.

In the apparatus, preferably, the integrated separation device further comprises a stirrer for mixing the cell culture solution with the fat-soluble material extracting solvent.

In the apparatus, preferably, the cell concentration device, the cell maturation device, the mixing device, and the separation device are integrated into a single device.

### Advantageous Effects

According to the present invention, intact cells and fat-soluble materials can be produced at a low cost and high efficiency from a culture of cells containing fat-soluble materials.

In producing cells and fat-soluble materials by cell culture according to the present invention, cultured cells are fermented to increase the content of fat-soluble materials, and modified such that a fat-soluble extracting solvent can readily penetrate into the cells and dissolve intracellular fat-soluble materials, thereby improving the production efficiency of intact cells and fat-soluble materials.

According to the present invention, cells and fat-soluble materials can be produced at high efficiency from a culture of cells containing fat-soluble materials by concentrating and/or fermenting cultured cells.

### DESCRIPTION OF DRAWINGS

FIG. 1 is a schematic view illustrating an embodiment of the apparatus of the present invention.

FIG. 2 is a schematic view illustrating an embodiment of the separation device useful in the present invention.

FIG. 3 is a schematic view illustrating an embodiment of a gravitational settling device useful as the separation device of the present invention,

FIG. 4 is a schematic view illustrating another embodiment of the apparatus of the present invention,

FIG. 5 is a schematic view illustrating a further embodiment of the apparatus of the present invention,

FIG. 6 is a schematic view illustrating an embodiment of an integrated separation device useful in the present invention,

FIG. 7 is a graph showing the effect of the fat-soluble material extracting solvent on the growth of cells,

FIG. 8 is a graph showing the effect of the fat-soluble material extracting solvents (alkanes) and vibrational disintegration (VD) on the extraction of fat-soluble materials from cells.

FIG. 9 is GC-TOF-MS spectrum of the biodiesel extracted from cells according to the present invention.

### BEST MODE

A detailed description will be given of a method for and an apparatus of producing cells and fat-soluble materials according to the present invention. The following embodiments are given only to illustrate the present invention, but should not be construed to limit the present invention.

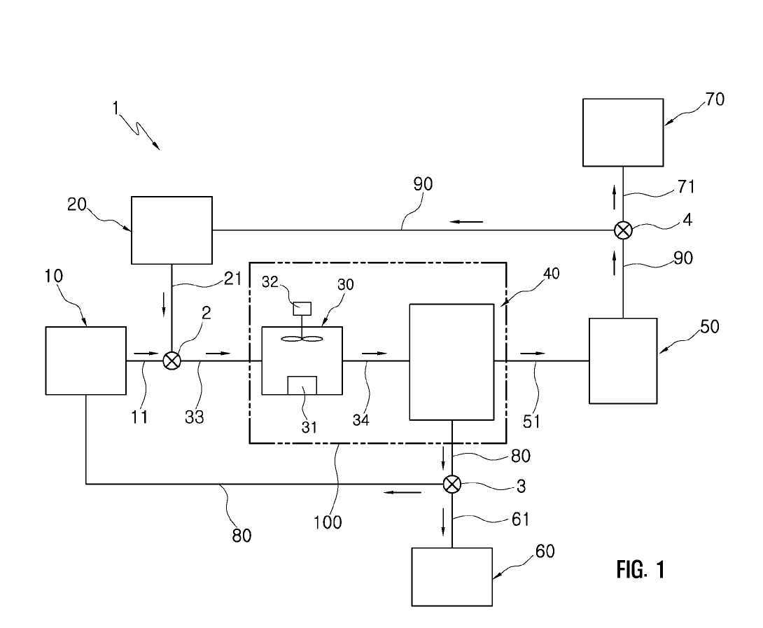

With reference to FIG. 1, a schematic view of an apparatus 1 according to the present invention is shown. As can be seen in FIG. 1, the apparatus 1 comprises a culturing device 10, a solvent device 20, a mixing device 30, a separation device 40, a fractionating device 50, a cell accommodating device 60, and a fat-soluble material-solvent accommodating device 70.

The culturing device 10 is adapted to culture cells containing fat-soluble materials such as microalgae and to feed the cell culture solution to a subsequent process. For use in the present invention, the culturing device 10 may be selected from among a wide spectrum of systems including ponds, artificial culturing facilities constructed on an open field, bioreactors, plastic bags, tubes, fermenters, shake flasks, and airlift columns. So long as it allows cells to grow, any type of culturing device may be employed in the present invention.

Cells in the culturing device 10 may be cultured in either an autotrophic manner or a heterotrophic manner, or in a mixotrophic manner where an autotrophic process is conducted prior to a heterotrophic process, or viceversa.

The solvent device 20 functions to store a solvent useful for extracting fat-soluble materials from cells in a cell culture solution, and to feed the solvent.

In the mixing device 30, the cell culture solution from the culturing device 10 and the fat-soluble material extracting solvent from the solvent device 20 are homogenously mixed with each other to exude the intracellular fat-soluble materials in cells of the cell culture solution into the fat-soluble material extracting solvent. Preferably, the cell culture solution is mixed at a ratio of about 5:1 with the fat-soluble material extracting solvent in the mixing device 30.

In a preferred embodiment, the mixing device 30 is equipped with a vibrational disintegrator 31 functioning to disassemble the cell culture solution. A preferred vibrational disintegrator 31 utilizes ultrasound. Given a vibrational impact, cell aggregates of the cell culture solution are separated into individuals which are more likely to exude out of the cells. In addition, when cell aggregates of he cell culture solution are separated into a smaller size aggregates or individual cells, they contact the fat-soluble material extracting solvent in a larger area, which leads to an improvement in the extraction efficiency of fat-soluble materials.

In combination with or instead of the vibrational disintegrator 31, a stirrer 32 may be installed in the mixing device 30 so as to facilitate the mixing of the cell culture solution with the fat-soluble material extracting solvent. The stirrer 32 functions to increase the contact of the cell culture solution with the fat-soluble material extracting solvent, thereby improving the extraction efficiency of fat-soluble materials.

According to a preferred embodiment, the cell culture solution of culturing device 10 and the solvent of the solvent device 20 are transferred in respective predetermined amounts into the mixing device 30 by a first peristaltic pump 2.

In this regard, a line 11 from the culturing device 10 and a line 21 from the solvent device 20 are connected to the first peristaltic pump 2, and a line 33 from the first peristaltic pump 2 extends to the mixing device 30.

The separation device 40 functions to perform continuous perfusion by applying, for example, an ultrasonic resonance field or gravitational settling to the mixed solution transferred from the mixing device 30 via the line 34, so as to concentrate, stagnate and separate the fat-soluble material-extracted cells from the mixed solution.

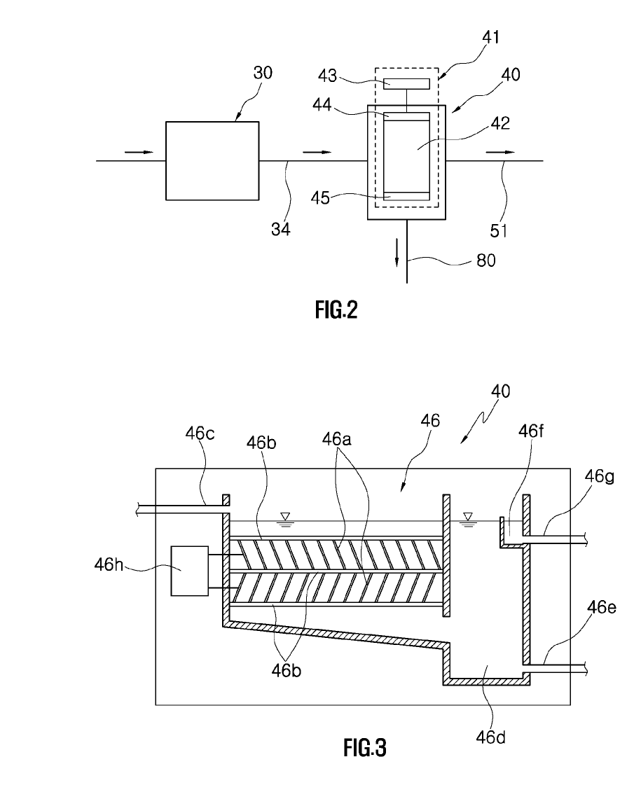

A filtration membrane device utilizing a filtration membrane in separating cells may be used as the separation device 40. Preferred is an ultrasonic resonance field generator 41 (FIG. 2) for separating cells by an ultrasonic resonance field or a gravitational settling device 46 (FIG. 3) for separating cells by gravitational settling.

When the filtration membrane device is employed as the separation device 40, it may be interchangeably installed with a microfiltration (MF) membrane, an ultrafiltration (UF) membrane, and a reverse osmosis (RO) membrane depending on the size of the cells to be separate.

Representative of the ultrasonic resonance field generator is an acoustic cell filter (Nature Biotechnology 12, 281-284 (1994)) of which the structure is illustrated in FIG. 2. The ultrasonic resonance field generator 41 is adapted to apply an ultrasonic resonance field to the mixed solution to aggregate or separate cells. With regard to the ultrasonic resonance field generator 41, reference may be made to U.S. Pat. No. 5,711,888 (issued on Jan. 27, 1998) titled ‘Multilayered piezoelectric resonator for the separation of suspended particles.’

As shown in FIG. 2, the acoustic cell filter 41 comprises an acoustic chamber 42, an ultrasonic generator 43, an ultrasonic transducer 44, and a reflector 45, and applies an ultrasonic resonance field to the mixed solution to induce the cells to form aggregates in the ultrasonic resonance field.

In the ultrasonic resonance field generator 41, like an acoustic cell filter, installed in the separation device 40, a first travelling wave is generated by the action of the ultrasonic generator 43 in cooperation with the ultrasonic transducer 44. When it is incident on the reflector 45, the first travelling wave from the ultrasonic transducer 44 is reflected in a backward direction to generate a second travelling wave. Accordingly, the first travelling wave generated from the ultrasonic transducer 44 collides with the second travelling wave propagating in the backward direction from the reflector 45 to produce a standing wave between the ultrasonic transducer 44 and the reflector 45. Two independent opposite waves combines to form a standing wave.

Typically, an ultrasonic standing wave can be formed in a structure where an ultrasonic transducer (e.g., a piezoelectric transducer) is positioned at a predetermined distance away from a reflector facing the transducer, or where two independent ultrasonic transducers are positioned at a predetermined distance away, facing each other.

An ultrasonic standing wave has a series of nodes and anti-nodes at fixed points along the transmission line. The nodes and anti-nodes account for a minimum and a maximum pressure amplitude, respectively, and individually appear twice every wavelength. In the presence of the ultrasonic resonance field, a position-dependent acoustic potential energy is created due to the discontinuity of particles, cells or droplets within the chamber. This phenomenon induces the cells to migrate into a position at which the acoustic potential is lowest, thereby entrapping the cells within the standing wave.

Accordingly, the cells are entrapped at the pressure nodes present every half wavelength, and finally congregate within the standing wave to form aggregates of cells.

The gravitational settling device 46 for use as the separation device 40 is designed to aggregate, stagnate and separate cells by utilizing a difference in sedimentation velocity between cells and media, the slope of the tube through which a solution flows, and an electromagnetic vibrator. The gravitational settling device 46 may be a cell settler (Biotechnology Solutions, USA).

With reference to FIG. 3, a gravitational settling device 46 is illustrated. The gravitational settling device 46 is designed to have a plurality of slope plates 46a arranged at regular intervals of distance in multiple layers. In the embodiment shown in FIG. 3, the slope plates 46a are arranged in two layers and supported by upper, middle, and lower shelf frames 46b. The slope plates 46a in the upper frame are arranged at a crossing angle with respect to those arranged in the lower frame, with the aim of reducing the flow rate to increase the sedimentation efficiency of cells.

The mixed solution of the cell culture solution and the fat-soluble material extracting solvent in the mixing device 30 is introduced via an inlet 46c connected to the line 34 into the gravitational settling device 46 where a settling process is performed. At the end of the arrangement of the slope plates 46, a cell collection chamber is provided for collecting and discharging settled cells. The cells collected in the cell collection chamber 46d are discharged externally through a cell outlet 46e. When it flows over a discharge way 46f, the solution from which settled cells are removed is discharged via a solution outlet 46g.

Preferably, the gravitational settling device 46 is equipped with a vibrator 46h to roll the slope plates 46a from side to side to effectively separate aggregated or stagnated cells.

Although the mixing device 30 and the separation device 40 are illustrated as separate structures in FIG. 1, they may be integrated into a single reaction device 100 as indicated by dashed two-dotted lines.

Turning to the fractionating device 50, it is designed to fractionate the cell-free solution transferred from the separation device 40 via a line 51 into a fat-soluble material-solvent (layer) (the fat-soluble material extracting solvent in which fat-soluble materials are dissolved) and water. That is, the solution from the separation device 40 is divided into an upper layer of fat-soluble material-solvent and a lower layer of water in the fractionating device 50.

As will be elucidated later, the cells separated in the separation device 40 are delivered to the cell accommodating device 60 and used in a subsequent process, while the upper layer of the fat-soluble material-solvent fractionated in the fractionating device 50 is transferred to the fat-soluble material-solvent accommodation device 70 and used in a subsequent process.

Optionally, a cell circulation line 80 may be provided, through which the cells separated as a lower layer in the separation device 40 are circulated to the culturing device 10. In a preferred embodiment, the cells of the separation device 40 are transferred to the cell accommodating device 60 or re-provided to the culturing device 10 via the cell circulation line 80.

Optionally, a solvent circulation line may be provided, through which the fat-soluble material-solvent divided as an upper layer in the separation device 50 may be recycled into the solvent device 20. In a preferred embodiment, the fat-soluble material-solvent of the separation device 50 is transferred to the fat-soluble material-solvent accommodation device 70 or re-provided to the solvent device 20 through the solvent circulation line 90.

The recycled cells are cultured in the culturing device 10, and fed, together with a fresh fat-soluble material extracting solvent from the solvent device 20 or the recycled fat-soluble material-solvent, to the mixing device 30, mixed therein, and undergo an additional round of dissolution of fat-soluble materials in the fat-soluble material extracting solvent before being delivered to the separation device 40 and the fractionating device 50 wherein the cell separation and the fractionation of the fat-soluble material-solvent are respectively repeated.

Preferably, re-culture, re-separation, and re-fractionation of the cells and the fat-soluble material-solvent may be repeated one or more times until the density of cells reaches a predetermined level while the fat-soluble materials in the fat-soluble material-solvent are concentrated to a desired level.

The cell accommodating device 60 receives the cells from the separation device 40 after the cells undergo one round of separation in the separation device 40 or after the cells reach a desired density through one or more rounds of recycling. In the cell accommodating device 60, the cells are temporarily stored or are processed for a subsequent purpose.

The cell accommodating device 60 may temporarily accommodate the high-density cells before delivery to a device for conducting various processes of ethanol fermentation, butanol fermentation, or organic acid fermentation to produce various useful materials, including bio-compounds, medicines, health functional foods, biofuels, protein hydrolysates, etc., from the cells. Alternatively, the cell accommodating device 60 itself may be a site where the high-density cells undergo the processes.

When various fermentations, such as ethanol fermentation, are carried out in the cell accommodating device 60, for example, it may be a fermenter. When the cells temporarily stay in the cell accommodating device 60 before they are transferred to a separate fermenter, the cell accommodating device 60 may be a temporal storage device.

The fat-soluble material-solvent accommodation device 60 receives the fat-soluble material-solvent from the fractionating device 50 after the fat-soluble material-solvent undergoes one round of fractionation in the fractionating device 50 or after the fat-soluble materials of the fat-soluble material-solvent reach a desired density through one or more rounds of recycling. The fat-soluble material-solvent is temporarily stored or is processed for a subsequent purpose in the cell accommodation 60.

The fat-soluble material-solvent accommodation device 70 may accommodate the fat-soluble material-solvent before delivery to a device for conducting various processes of extracting fat-soluble materials from the fat-soluble material-solvent, and producing various useful materials, such as medicines, health functional foods, biodiesel, etc., from the extracted fat-soluble materials. Alternatively, the fat-soluble material-solvent accommodation device 70 itself may be a site where the fat-soluble material-solvent undergoes the processes.

When fat-soluble materials are extracted from the fat-soluble material-solvent, and biodiesel is produced from the extract in the fat-soluble material-solvent accommodation device 70, for example, the fat-soluble material-solvent may be a distillation-biodiesel production device. When the fat-soluble material-solvent temporarily stays in the fat-soluble material-solvent accommodation device 70 before it is transferred to a separate distillation-biodiesel production device, the fat-soluble material-solvent accommodation device 70 may be a temporal storage device.

Preferably, the apparatus of the present invention further comprises a second peristaltic pump 3 for selectively delivering the cells separated in the separation device 40 to the culturing device through the cell circulation line 80 or to the cell accommodating device 60 according to the density of the cells.

In this context, the second peristaltic pump 3 is provided on the cell circulation line 80 between the separation device 40 and the culturing device 10. An additional line 61 from the second peristaltic pump 3 is communicates with the cell accommodating device 60.

In a preferred embodiment, the second peristaltic pump 3 is operated, if the density of the cells is below a desired level in the separation device 40, to recycle the cells to the culturing device 10, so that the cells undergo one or more additional rounds of culturing, dissolution of fat-soluble materials in the fat-soluble material extracting solvent, and cell separation, and, if their density reaches a desired level in the separation device 40, to transfer the cells to the cell accommodating device 60.

Preferably, the apparatus 1 of the present invention further comprises a third peristaltic pump 4 for transferring the fat-soluble material-solvent fractionated in the fractionating device 50 to the solvent device 20 or the fat-soluble material solvent accommodation device 70 according to the concentration of the fat-soluble material-solvent. In this context, the third peristaltic pump 4 is provided on the solvent circulation line 90 between the fractionating device 50 and the solvent device 20, and communicates with the fat-soluble material-solvent accommodation device 70 via an additional line 71.

In a preferred embodiment, the third peristaltic pump 4 is operated, if the concentration of fat-soluble materials in the fat-soluble material-solvent is below a desired level in the fractionating device 50, to recycle the fat-soluble material-solvent to the solvent device 20, so that the fat-soluble material-solvent undergoes one or more additional rounds of fractionation, and, if the concentration reaches a desired level in the fractionating device 50, to transfer the fat-soluble material-solvent to the fat-soluble material-solvent accommodation device 70.

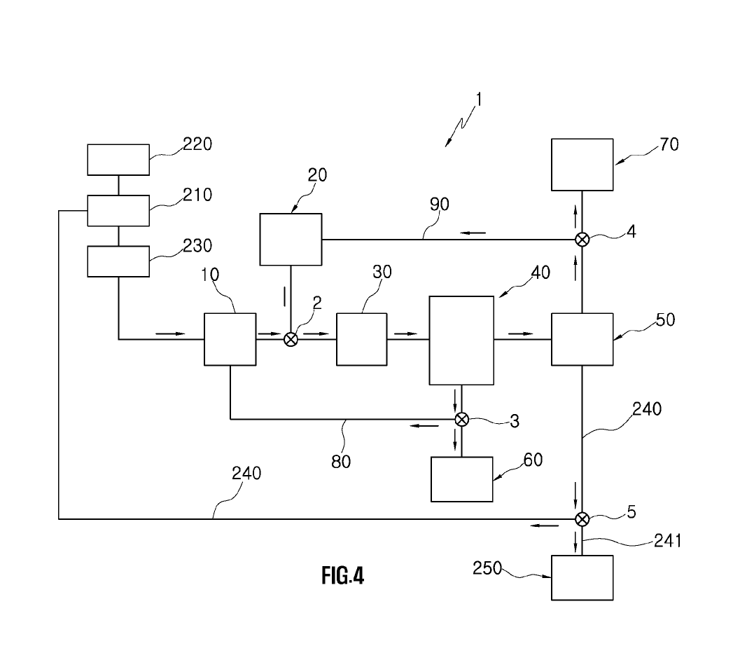

Referring to FIG. 4, the apparatus 1 of the present invention further comprises a fermenter 210, a biogas accommodation device 220, and an organic wastewater accommodation device 230. The organic wastewater accommodation device 230 feeds organic wastewater as the cell culture solution to the culturing device 10.

The fermenter 210 is a device in which an organic waste including carbohydrates, proteins, lipids, etc., like food waste, is fermented to produce organic wastewater and biogas.

In this regard, if captured, carbon dioxide generated in the fermenter 210 can be provided as a carbon source for the growth of cells (microalgae) in the culturing device 10.

The biogas accommodation device 220 is adapted to capture and store the biogas produced in the fermenter 210, such as hydrogen and methane, and optionally to process the biogas if necessary.

The organic wastewater accommodation device 230 is provided for storing the organic waste (e.g., fermented liquid) generated after fermentation in the fermenter 210.

Although the apparatus 1 of the present invention of FIG. 4 is illustrated to form a plant comprising the fermenter 210, the biogas accommodation device 220 and the organic wastewater accommodation device 230, together with the culturing device 10, it should be appreciated that a system in which the organic wastewater accommodation device 230 is established remotely from the culturing device 10 and provides organic wastewater to the culturing device 10 also falls within the scope of the present invention.

A water circulation line 240 is provided between the fractionating device 50 and the fermenter 210, through which the water separated in the fractionating device 50 is recovered to the fermenter 210.

The water separated in the fractionating device 50 can be transferred to the water accommodation device 250 and stored therein until reuse for a desired purpose. In this regard, a fourth peristaltic pump 5 is provided on the water circulation line 240 to selectively feed water from the fractionating device 50 to the water accommodation device 250 or the fermenter 210.

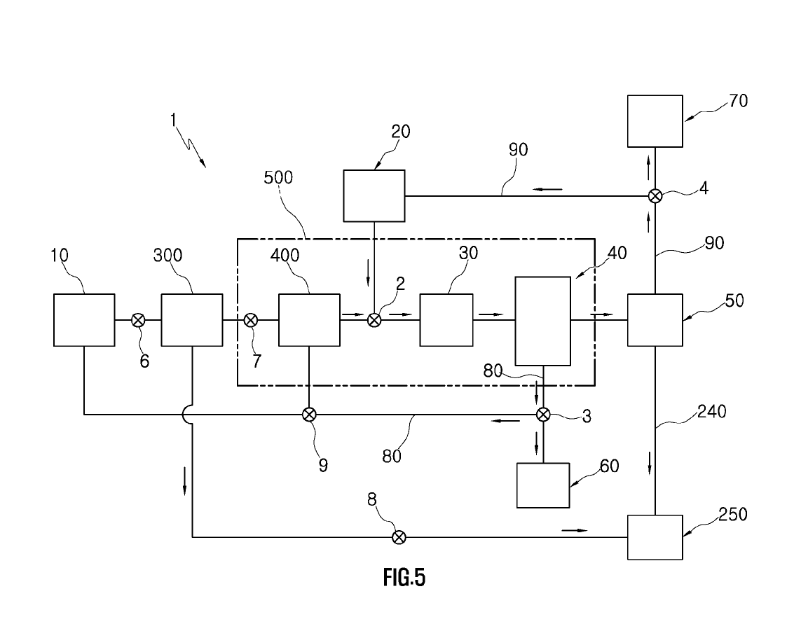

The apparatus 1 of the present invention, as shown in FIG. 5, may further comprise a cell concentration device 300 for concentrating cells of the cell culture solution, and a cell maturation device 400 for maturing the cells between the culturing device 10 and the mixing device 30.

The cells of the cell culture solution fed from the culturing device 10 are concentrated in the cell concentration device 300.

The filtration membrane, the ultrasonic resonance field, or the gravitational settling delineated in conjunction with the separation device 40 of FIG. 1 can be applied to the cell concentration device 300 so as to concentrate the cells of the cell culture solution transferred from the culturing device 10 by separating the cell culture solution into cells and an aqueous solution.

The cell maturation device 400 is adapted to mature the concentrated cells transferred from the cell concentration device 300.

In the cell maturation device 400, for example, the cells are matured by feeding a cell maturation solution which is free of limiting nutrients but contains a carbon source, thus increasing content of fat-soluble material in cells, and are modified such that the fat-soluble material extracting solvent readily penetrates into the cells to dissolve fat-soluble materials.

After being matured in the cell maturation device 400, the cell culture solution is transferred to the mixing device 30 while the fat-soluble extracting solvent of the solvent device 20 is fed to the mixing device 30 so as to extract fat-soluble materials from cells.

A fifth peristaltic pump 6 is provided between the culturing device 10 and the cell concentration device 300 to transfer the cell culture solution in a predetermined amount to the cell concentration device 300.

Likewise, a sixth peristaltic pump 7 is provided between the cell concentration device 300 and the cell maturation device 400 to flow the cell culture solution in a predetermined amount into the cell maturation device 400.

The cell concentration device 300 communicates with the water accommodation device 250 through the water circulation line 310 so that the water collected in the cell concentration device 300 is allowed to flow to the water accommodation 250 and is reused in subsequent processes. The water circulation line 310 is equipped with a seventh peristaltic pump 8.

Through the cell circulation line 80, the cells separated in the separation device 40 are transferred to the cell accommodating device 60 or delivered selectively to the culturing device 10 or the cell maturation device 400. In this regard, an eighth peristaltic pump 9 is provided on the cell circulation line 80 to control the delivery of the cells separated in the separation device 40 to the culturing device 10 or the cell maturation device 400.

Although the cell maturation device 400, the mixing device 30 and the separation device 40 are illustrated as separate structures in FIG. 5, they may be assembled into an integrated separation device 500 as indicated by dashed two-dotted lines.

That is, the integrated separation device 500 is designed to perform the respective steps of maturing the concentrated cells transferred from the cell concentration device 300, mixing the matured cell culture solution with the fat-soluble material extracting solvent from the solvent device 20, and separating the cells from the mixed solution in a single process.

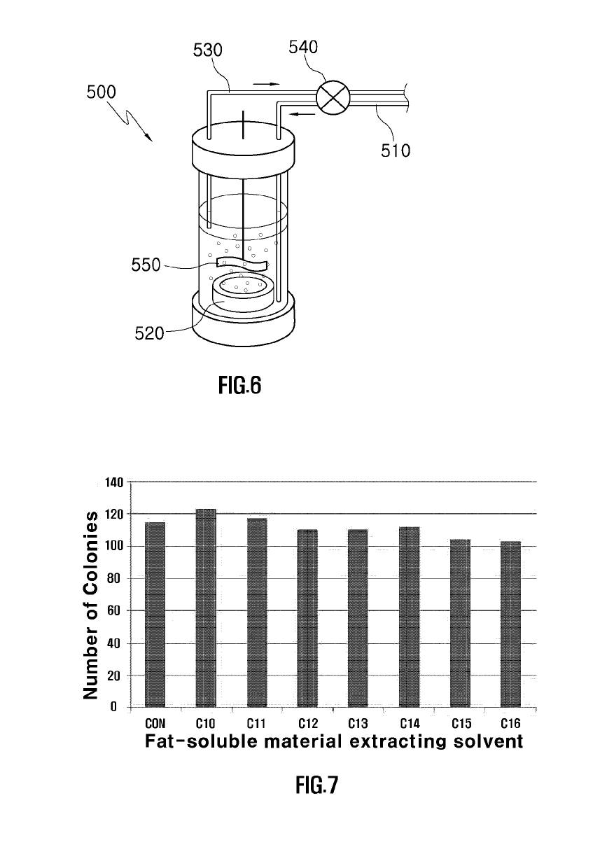

With reference to FIG. 6, there is an embodiment of the integrated separation device 500 in which the cells are matured by adding a cell maturation solution, mixed with a fat-soluble material extracting solvent to exude intracellular fat-soluble materials into the solvent, and separated as a lower layer from the upper layer of water and fat-soluble material-solvent.

In more detail, the integrated separation device 500 comprises an inlet line 510, a spout tube 520, a recovery line 530, a peristaltic pump 540 and a stirrer 550. The inlet line 510 is provided for feeding the fat-soluble material extracting solvent from the solvent device 20 to the integrated separation device 500. The spout tube 520 is connected to the end of the inlet line 510 to spout the fat-soluble material extracting solvent upward from the bottom of the cell culture solution. The fat-soluble material extracting solvent of the integrated separation device 500 plus fat-soluble materials dissolved in the fat-soluble material extracting solvent are recovered to the solvent device 20 through the recovery line 530. The peristaltic pump 540 is located on both the inlet line 510 and the recovery line 530, functioning to provide a pressure necessary for transferring the solvents (the fat-soluble material extracting solvent and the fat-soluble material-solvent). The stirrer 550 functions to mix the cell culture solution with the fat-soluble material extracting solvent in the integrated separation device 500.

By the action of the integrated separation device 500, both the fat-soluble material extracting solvent and the fat-soluble material-solvent are recycled between the integrated separation device 500 and the solvent device 20, repeatedly dissolving and exuding intracellular fat-soluble materials out of the matured cells. When the concentration of the fat-soluble material reaches a predetermined level, the recycling of the solvents ceases to allow layer separation into a lower cell layer and an upper water and fat-soluble material-solvent layer in the integrated separation device 500.

The separated lower cell layer, as described in conjunction with FIG. 1, is recovered to the cell accommodating device 60 or recycled to the culturing device 10 while the upper water and fat-soluble material-solvent layer is transferred to the fractionating device 50 wherein the fractionation elucidated in FIG. 1 is conducted.

1. Fermentation of Organic Waste (Food Waste)

An organic waste (e.g., food waste) is subjected to semi-anaerobic or aerobic hydrolysis/acidogenic fermentation in a fermenter 210 (FIG. 4) to generate organic wastewater and biogas, and the organic wastewater collected in the organic wastewater accommodation device 230 is transferred to the culturing device 10 to culture microalgae (cells) while the biogas captured in the biogas accommodation device 220 serves as an energy source.

The fermenter 210 can maintain the fermentation temperature at 45° C. The microorganisms (cells) employed for the fermentation are strains capable of degrading organics, such as carbohydrates, proteins, and lipids, and are summarized in Table 1, below.

TABLE 1

| Fermentation Process | Strain | Degradable target |
|---|---|---|
| Semi-anaerobic | Cellulomonas cellulans | cellulose, chitin, pectin |
| hydrolysis/acidogenic | Flavobacterium breve | cellulose |
| fermentation | Bacillus | carbohydrates |
| | amyloliquefaciens | |
| | Bacillus licheniformis | proteins |
| | Bacillus subtilis | carbohydrates, proteins |
| | Bacillus alcalophilus | lipids |
| Anaerobic acidogenic | Clostridium | sugars, amino acids, |
| fermentation | acetobutyricum | long-chain fatty acids |
| | Clostridium butyricum | |
| Anaerobic methane | Methanogenic microbes | acetate, formate |
| fermentation | | |

Food waste is mixed at a ratio of 1:1 with water, and finely ground using a grinder to facilitate the fermentation by microorganisms. After fermentation for 2 days in a semi-anaerobic condition, the fermented liquid (organic wastewater) is drained out and introduced into the organic wastewater accommodation device 230 using a pump. The organic wastewater generated in the fermenter 210 is allowed to flow into the bottom of the organic wastewater accommodation device 230 and stay therein. In the organic wastewater accommodation device 230, COD (chemical oxygen demand), nitrogen and phosphorus levels of the organic wastewater are measured and adjusted into suitable levels for the growth of cells.

After the semi-anaerobic hydrolysis/acidogenic fermentation of food wastes, the resulting organic wastewater may be directly used in culturing microalgae. It may be further subjected to secondary anaerobic hydrolysis/acidogenic fermentation to produce hydrogen and organic wastewater. Alternatively, the organic wastewater may be used in culturing microalgae only after tertiary methane fermentation by methanogens.

In these fermentation processes, biogases such as hydrogen and methane are generated, and they can be captured and stored in the biogas accommodation device 220 until use for suitable purposes.

2. Cell Culture Solution (High-Density)

The cells cultured in the culturing device 10 include plant cells, fungi, diatoms, dinophyceae, haptophytes, blue-green algae, rhodophyceae, chlorophyceae, and prokaryotes.

For example, Chlorella protothecoides may be used in the present invention. C. protothecoides can be cultured at a density 10-fold higher than most microalgae, and is very profitable in view of the productivity of biomass. In an ideal heterotrophic condition, C. protothecoides allows the production of biomass at a rate of up to 35 gfw/L, with fat-soluble materials accounting for about 55% of the biomass.

Assuming that cells produce fat-soluble materials at a relatively constant rate, a higher biomass density naturally leads to a greater total amount of produced valuable materials per volume. Conventional fermentation methods for growing cells guarantee the production of biomass at a density of from about 50 to about 80 g/L or less.

The cells used in the present invention have a biomass density of at least about 100 g/L, preferably at least about 130 g/L, more preferably at least about 150 g/L, far more preferably at least about 170 g/L, and most preferably at least 200 g/L.

Therefore, although the production rate of valuable materials in the cells is slightly reduced at such a high biomass density, the entire production rate of valuable materials per volume is significantly higher, compared to conventional methods.

In the method according to a preferred embodiment of the present invention, cells are re-cultured after cell culturing, the mixing of the primary cell culture solution and the fat-soluble material extracting solvent, and the concentration/separation of cells, and the re-cultured cells are mixed again with the fat-soluble material extracting solvent or the fat-soluble material-solvent (fat-soluble materials dissolved in the fat-soluble material extracting solvent) and subjected to aggregation and separation, repeatedly two or more times until the cells of the cell culture solution are grown to a desired density to ensure high productivity.

C. protothecoides can grow under heterotrophic conditions on glucose or corn stover hydrolysate (CSH). Heterotrophic growth can increase the content of fat-soluble materials while decreasing the direct dependence on solar energy. The biodiesel produced by C. protothecoides is substantially identical in energy density to petroleum-based diesel.

Chlorella is easy to engineer using a molecular biological method, and is possible to culture at an increased CO₂ concentration in a large-scale photobioreactor.

3. Disposal of Organic Wastewater by Microalgae (Cells)

Typical wastewater contains a carbon source at a lower percentage than a nitrogen source. Conventional disposal methods of wastewater using microorganisms are poor in nitrogen removal rate. Activated sludge methods can lower BOD (biological oxygen demand) by 90% or higher, but nitrogen only by 20˜50% from wastewater. For this reason, the C/N ratio is intentionally increased by providing an organic carbon source such as ethanol or glucose in order to remove nitrogen. If wastewater is released into a river without sufficiently removing nitrogen therefrom, it may cause a red tide, disturbing the ecosystem as well as provoking economic damage.

In a preferred embodiment of the present invention, organic wastewater containing a sufficient amount of an organic carbon source, such as fermented food wastewater or fermented livestock wastewater, are adjusted with regard to levels of organic materials and nitrogen components, and disposed by culturing microalgae therein.

When microalgae are cultured under heterotrophic or mixotrophic conditions, they require an organic carbon source, carbon dioxide, water, and nitrogen. Because organic carbon and nitrogen are fed from organic wastewater containing a sufficient amount of an organic carbon source (e.g., fermented liquid of organic waste, particularly, food waste) thereto, the microalgae grow actively under heterotrophic or mixotrophic conditions, correspondingly removing organic carbon sources, nitrogen and phosphorus from the organic wastewater (fermented liquid of livestock wastewater or food waste).

For use in heterotrophic or mixotrophic fermentation of microalgae, the organic wastewater is preferably diluted to the extent of a TCOD (total chemical oxygen demand) of 100 to 10,000 mg/l and a nitrogen concentration of 100 to 800 mg/l, with pH of 5.0˜5.5 maintained.

To promote the growth of microalgae, inorganic matter may be added. The inorganic matter useful in the present invention may be selected from the group consisting of Mg²⁺, Ca²⁺, and phosphorus. The growth of microalgae can be further enhanced by adding Mg²⁺ or Ca²⁺ to the organic wastewater and controlling the ratio of nitrogen and phosphorus, resulting in increasing the disposal efficiency of the organic wastewater. In the organic wastewater, Mg²⁺ may be present at a concentration of from 100 to 1,000 mg/l, preferably at a concentration of from 200 to 700 mg/l, and more preferably at a concentration of from 300 to 500 mg/l. The organic wastewater contains Ca²⁺ at a concentration of from 10 to 300 mg/l, preferably at a concentration of from 50 to 200 mg/l, and more preferably at a concentration of from 100 to 150 mg/l.

Phosphorus is added to the organic wastewater in such a way that the ratio of nitrogen to phosphorus ranges from 20:1 to 3:1, preferably from 15:1 to 5:1, and more preferably from 12:1 to 10:1.

Metal ions indispensible for the growth of microalgae, such as phosphorus, iron, zinc, copper, and aluminum, may not be added to an organic wastewater if it is disposed with activated sludge. Nonetheless, these ions are helpful in culturing the microalgae at a high density.

4. Maturation of Cells

When intact cells and fat-soluble materials are produced only from a cell culture solution using a fat-soluble material extracting solvent, it is somewhat difficult to obtain fat-soluble materials from the cells due to a change in the content of fat-soluble materials with the species and strain of culture cells or microorganisms, and diversity of the cell permeability of the fat-soluble material extracting solvent, but cell maturation can mitigate or overcome these problems.

Preferably, the cell maturation process of the present invention increases the intracellular fat-soluble material content to improve the productivity of fat-soluble materials, and alters the cells in such a way that the fat-soluble material extracting solvent readily penetrates into the cells and exudes fat-soluble materials out of cells.

For this, a carbon source and a limiting nutrient are added to the cells in a nutrient medium at a sufficiently high rate to increase the density of biomass in the cell culture solution.

As used herein, the term “limiting nutrient” refers to a nutrient source (inclusive of a nutrient itself) indispensible for the growth of cells in view of the fact that the depletion of a limiting nutrient from the cell culture solution substantially limits the growth or replication of cells.

However, because the other nutrients are still abundant, the organisms can continue to produce and accumulate intra- and (or) extracellular products. In this regard, the limiting nutrient determines the kind of products accumulated, and the property of the cells. Therefore, the provision of a limiting nutrient source at a specific rate can regulate the growth rate of cells and the production or accumulation of a desired product (for example, lipids).

A biotechnological batch process which is based on feeding of at least one substrate (e.g., a carbon source and a growth limiting nutrient) to a culture is called fed-batch culture. If the substrate is a carbon source that already sufficiently exists (e.g., at about 200 g/L or higher per a biomass density of 60 g/L) within the cells, its addition is known to have a harmful effect on the cells. If too excessive, a carbon source is known to cause a harmful effect (inclusive of osmosis stress) on the cells and suppress the early productivity of cells.

In the present invention, a sufficient amount of substrates is provided to achieve a desired biomass density of cells without causing undesirable harmful effects.

To grow cells, the present invention may further comprise a biomass density enhancer. It has a primary aim of increasing the biomass density within the cell culture solution.

The addition rate of a carbon source is maintained within such a range in which no significant harmful effects on the survival of cells are evoked. A suitable quantity range of carbon sources necessary for specific cells during a fermentation process is known to those skilled in the art.

Preferably, the carbon source used in the present invention is a non-alcoholic carbon source, that is, a carbon source containing no alcohols. The term “alcohol” as used herein refers to a compound containing up to 4 carbon atoms with one hydroxy group. For example, methanol, ethanol and isopropanol may be used. For the purpose of the present invention, hydroxylated organic acids, for example, lactic acid, and analogs thereof are regards as non-alcoholic carbon sources. More preferably, the carbon source of the present invention includes fructose, glucose, sucrose, molasses, and starch, but is not limited thereto.

Typically and preferably, corn syrup may be used as a primary carbon source. Hydroxy fatty acids, triglycerides, and fatty acids in di- and monoglyceride can also act as a carbon source.

Urea, nitrates, nitrites, bean proteins, amino acids, proteins, corn steep liquor, yeast extract, animal by-products, and inorganic ammonium salts are used as useful nitrogen sources. More preferred are ammonium salt of sulfide and hydroxide. The most preferable is ammonium hydroxide.

Among other limiting nutrient sources are a carbon source, a phosphate source, a vitamin source (e.g., vitamin B12, pantothenate, thiamine), a trace metal source (e.g., zinc, copper, cobalt, nickel, iron, manganese, and molybdenum), and a major metal source (e.g., magnesium, calcium, sodium, potassium, and silica). The trace metal source and the major metal source may include a sulfate and chloride of their own metals (non-limiting examples include MgSO₄.7H₂O; MnCl₂.4H₂O; ZnSO₄.7H₂O; CoCl₂.6H₂O; Na₂MoO4.2H₂O; CuSO4.5H₂O; NiSO4.6H₂O; FeSO₄.7H₂O; CaCl₂ K₂SO₄ KCl; [symbol] Na₂SO₄).

When ammonium is used as a nitrogen source, the fermentation medium becomes acidic unless it is adjusted with a base or a buffer. Ammonium hydroxide as a primary nitrogen source may be used for pH control. Cells will grow over a wide range of pH, for example, pH of from 5 to 11. A suitable pH range for the fermentation of specific microorganisms is known in the art.

The method for growing the cells in accordance with the present invention may comprise a production phase. In this production phase, the primary use of substrates by microorganisms does not lead to an increase in biomass density, but to the production of lipids. Although lipids are also produced by cells during a biomass density increase phase, the primary goal of this phase is to increase the biomass density as described above. Typically, the limiting nutrient source is added in a smaller amount, and preferably is not added during the production phase.

As the level of dissolved oxygen is reduced during the production phase, the production rate of lipids drastically increases. Therefore, the level of dissolved oxygen in the fermentation medium during the biomass density increase phase is preferably at least about 8% of saturation, and more preferably at least about 4% of saturation, whereas the level of dissolved oxygen in the fermentation medium during the production phase is on the order of up to 3% of saturation, preferably on the order of up to 1% of saturation, and more preferably on the order of 0% of saturation. The level of dissolved oxygen may be at or around a saturation point in the early phase of fermentation, and decreases to the predetermined points as the microorganisms grow.

In the present invention, the level of dissolved oxygen in a cell culture solution may be changed during a culturing process. In a culturing process with a total culture time of about 90 to 100 hrs, for example, the level of dissolved oxygen in the cell culture solution may be maintained at about 8% for a first 24 hrs of culturing, at about 4% during a period of from a time point of 24 hrs to a time point of 40 hrs after culturing, and at about 0.5% or less during a period of from a time point of 40 hrs after culturing to the end of culturing.

The level of dissolved oxygen in the cell culture solution may be controlled by adjusting the amount of oxygen in the reactor, or preferably by adjusting the speed at which the cell culture solution is stirred. For example, the level of dissolved oxygen in the cell culture solution is higher at a higher stirring speed than a lower stirring speed. A range of stirring speeds necessary for achieving a predetermined level of dissolved oxygen in the cell culture solution can be readily determined by a person having ordinary skill in the art.

A temperature at which the culturing process of the present invention is carried out is at least about 20° C., preferably at least about 25° C., and most preferably at least about 30° C. It will be understood that cold water retains a higher level of dissolved oxygen than warm water. Therefore, higher temperatures of the cell culture solution have an additional advantage of decreasing the level of dissolved oxygen.

Certain cells may need a certain amount of salts or minerals in their culture medium. The salts or minerals, particularly chlorine ions, may erode a fermenter or other downstream facilities. To prevent or reduce the undesired effect of abundant chlorine ions present in the culture medium, the method of the present invention comprises using chlorine-free sodium salts as a sodium source in the culture medium, preferably, sodium sulfate. Particularly, the chlorine-free sodium salt covers a significant portion of sodium demand. For example, sodium chloride is provided in such an amount so as to account for less than about 75% of the sodium level required by the cell culture solution, preferably less than about 50%, and more preferably less than about 25%. According to the present invention, cells (microorganisms) may be cultured at a chloride concentration of less than 3 g/L, preferably at a chloride concentration of less than about 500 mg/L, more preferably at a chloride concentration of less than about 250 mg/L, and far more preferably at a chloride concentration of from about 60 mg/L to about 120 mg/L.

Examples of the chlorine-free sodium salts useful in the present invention include a mixture of soda ash (sodium carbonate) and sodium oxide, sodium carbonate, sodium bicarbonate, sodium sulfate, and a combination thereof, with preference for sodium sulfate. Because soda ash, sodium carbonate and sodium bicarbonate are prone to increasing the pH of the cell culture solution, pH adjustment is needed when they are used. Sodium sulfate is used in such an amount so as to meet the demand of cells for sodium. The effective sodium concentration (in g/L of Na) is at least about 1 g/L, and preferably ranges from about 1 g/L to about 50 g/L, and more preferably from about 2 g/L to about 25 g/L.

5. Extraction of Fat-Soluble Materials from Cells

A process of harvesting cells from a large volume of cell culture solution is predominantly responsible for the cost generated for the production of biomass and useful materials from cells. In conventional methods of producing fat-soluble materials, the expense paid for the process of extracting fat-soluble materials by harvesting cells from a cell culture solution, drying and disrupting the cells accounts for 40˜60% of the total production cost.

Because cells are destroyed during the extraction of fat-soluble materials in conventional methods, biomolecules other than fat-soluble materials are difficult to harvest. The present invention can overcome this problem by recycling cells at a low cost without destruction.

In addition to being highly selective for fat-soluble materials and biocompatible, the fat-soluble material extracting solvent used in the present invention should contact cells without exerting significant damage to cellular activity. Generally, the solvent has an octanol/water partition coefficient [log Poct] of 5 or higher (except for dodecanone). Of the solvents with a log Poct of 4-5, hexane and heptanes are toxic to cells while decanol and dipentyl ether are harmless.

Examples of the fat-soluble material extracting solvent available in the present invention includes 1,12-dodecanedioic acid diethyl ether, n-hexane, n-heptane, n-octane, n-dodecane, dodecyl acetate, decane, dihexyl ether, isopar, 1-dodecanol, 1-octanol, butyoxyethoxyethane, 3-octanone, cyclic paraffins, varsol, isoparaffins, branched alkanes, oleyl alcohol, diethylether, and 2-dodecane.

The fat-soluble material extracting solvent useful in the present invention may contain at least one C4-C16 hydrocarbon, preferably, C10, C11, C12, C13, C14, C15 or C16 hydrocarbon.

Ultrasonic irradiation on microorganisms by the vibrational disintegrator 31 without cell damage is dose dependent at a low frequency. Microorganisms survive for a long period of time at irradiation of high frequencies. A study on the effects of irradiation time and frequencies within a range (20 kHz-1 MHz) which has no influence on cellular activity and allows the optimal extraction of fat-soluble materials demonstrates that the extraction efficiency is influenced by various different frequencies, intensities, and exposure times.

Using frequencies in a suitable range (20 kHz-60 kHz) at appropriate intensities for different periods of time, fat-soluble materials can be optimally extracted without causing damage to cells. That is, frequency, intensity and exposure time are factors which have influences on the extraction of fat-soluble materials. Since interaction between cells and ultrasound is complicatedly influenced by cell sizes, cell morphology, cell wall compositions and physiological conditions, ideal frequencies for extracting fat-soluble materials can be selected from among various frequencies of 20 kHz, 1 MHz, 20-100 kHz, 20-60 kHz, 30-50 kHz, and 40 kHz. An appropriate combination of the fat-soluble material extracting solvent and the vibrational disintegration enables fat-soluble materials to be extracted at a yield of almost 100% (10% of total cellular fatty acids).

When a stirrer 32 is used instead of vibrational disintegration, it increases (improves) the contact of the cell culture solution with the fat-soluble material extracting solvent, making a contribution to an improvement in extraction efficiency, like vibrational disintegration. The stirrer can be used in combination with the vibrational disintegration.

6. Cell Separation without Cell Damage

In order to produce fat-soluble materials while recovering cells without damage, conventional methods utilize the natural sedimentation of cells by gravity after dissolving intracellular fat-soluble materials. On a mass scale, however, the natural sedimentation of cells requires a long period of time. The present invention overcomes this problem by utilizing a mechanical device in separating cells.

As the separation device 40, a continuous perfusion based device that allows cells to be separated elaborately and effectively without damaging cells can be used. A filtration membrane device may be used as the separation device 40. However, the ultrasonic resonance field device 41 utilizing an ultrasonic resonance field or the gravitational settling device 46 utilizing a gravitational settling strategy is preferred.

For instance, when the ultrasonic resonance field generator 41 is applied as the separation device 40, the suitable use of important factors including ultrasonic frequencies in the fat-soluble material extracting solvent and the ultrasonic resonance field, and the distance between the ultrasonic transducer 44 and the reflector 45 allows fat-soluble materials to be extracted at an extraction efficiency of almost 100% (10% of total cellular fatty acids).

In the processes of mixing and contacting the fat-soluble material extracting solvent with the cell culture solution and separating cells, pathogens present in the cell culture solution and fat-soluble growth inhibitors secreted by cells can be eliminated, which, in turn, enables the cells to be effectively cultured at a high density.

Chlorella vulgaris secretes chlorellin, a fat-soluble growth inhibitor (Pratt et al., (1944) Science. 28; 99 (2574):351-2.).

As for Botryococcus braunii, its hydrocarbon products strongly adhere to the envelope of the strain. Since stirring alone does not guarantee sufficient contact between the cell culture solution and the fat-soluble material extracting solvent, it is difficult to recover the hydrocarbons at high efficiency by stirring alone. The extraction efficiency can be increased by improving contact between the organic solvent and the cell culture solution, as in the processes of mixing and contacting the fat-soluble material extracting solvent with the cell culture solution and separating cells in accordance with the present invention.

7. Biodiesel Production

The production of biodiesel is largely classified into direct use, supercritical fluid extraction, and transesterification. When animal and vegetable oils are directly used as biodiesel, a mixture of diesel and the biodiesel increases in viscosity with time, imparting a burden on the engine. Supercritical fluid extraction exhibits rapid reaction speeds, and if applied to fatty acids and glycerin, exerts complete thermal decomposition without the production of by-products.

Transesterification, generally used thus far, is a process in which a triglyceride is reacted with an alcohol in the presence of a catalyst to produce a fatty acid ester and glycerin. The separated fatty acid ester is used as biodiesel. For transesterification, there are a chemical method using a chemical catalyst, and an enzyme method using a biocatalyst.

The chemical method is employed in most processes because the catalyst is inexpensive and allows a high conversion rate within a short time. Typically, acid catalysts (HCl, H₂SO₄, etc.) or base catalysts (NaOH, KOH, etc.) are employed. An acid catalyst is suitable for the conversion of waste oils rich in free fatty acids, but suffers from the disadvantage of eroding facilities, and requiring large energy consumption due to high temperature and high pressure. A base catalyst is found in many commercial processes because it is higher in reaction rate than the acid catalyst. An enzyme method uses lipase as a catalyst for esterification. It requires a lesser amount of alcohol, compared to the chemical method, and omits a process of separating and purifying the glycerin. In addition, the enzyme method can esterify all fatty acids present in the oil, with the production of highly pure products. To overcome drawback of the enzyme method, the enzyme activity is maintained using a porous hollow fiber membrane into which a hydrophilic anionic exchange group is introduced after a graft chain is formed by radiation induced grafted polymerization thereon.

Diesel reactors applicable to diesel production may be a batch type or a continuous fed type according to process, and may be divided into a catalyst reactor, an annular reactor and a tubular reactor according to morphology.

Preferably, biodiesel is extracted from the fractionated lipids using a chemical transesterification method in the present invention.

8. Ethanol Production

On the whole, raw materials of ethanol include glucoses (sugar cane, sugar beet), starches (corn, potato, sweet potato), and lignocelluloses (woods, rice straw, waste paper). Glucoses can be converted into ethanol through a fermentation process immediately after a simple pretreatment. However, starches and lignocelluloses are subjected to suitable pretreatment and saccharification processes to produce saccharified liquid from which ethanol is produced by a fermentation process.

In the present invention, the cells concentrated in the fractionation device 50 can be used, on the basis of the ability of Clostridium phytofermentans (e.g., ACCT 700394^{T}), an aerobic cellulose degrading bacterium, in fermentation to produce ethanol and hydrogen (without pretreatment, saccharification and ethanol fermentation).

Anaerobic cellulose degrading bacteria can be isolated from various habitats (e.g., soil, deposits, swamps, mammal intestines, etc.) (Madden, et al., (1982) Int J Syst Bacteriol 32, 87-91; Murray et al., (1986) Syst Appl Microbiol 8, 181-184; He et al., (1991) Int J Syst Bacteriol 41, 306-309; Monserrate et al., (2001) Int J Syst Evol Microbiol 51, 123-132.).

Clostridium phytofermentans is a long, thin, straight, rod-shaped cell having motility. It forms a spherical spore (0.9-1.5 μm in diameter). Other features of Clostridium phytofermentans are disclosed in Warnick et al., Int. J. Systematic and Evol. Microbiology, 52, 1155-1160 (2002).

Clostridium phytofermentans can ferment a wide spectrum of materials into fuels at high efficiency. Interestingly, the bacterium can ferment waste, for example, lactose, waste paper, leaves, herbs, wood chips, and/or sawdust (Korean Patent Laid-Open Publication No. 10-2008-0091257).

Clostridium phytofermentans may be used alone or in combination with at least one of yeasts, fungi (e.g., Saccharomyces cerevisiae, Pichia stipitis, Trichoderma species, and Aspergillus species), or other bacteria (e.g., Zymommonas mobilis, Klebsiella oxytoca, Escherichia coli, Clostridium acetobutylicum, Clostridium beijerinckii, Clostridium papyrosolvens, Clostridium cellulolyticum, Clostridium josui, Clostridium termitidis, Clostridium cellulosi, Clostridium celerecrescens, Clostridium populeti, and Clostridium cellulovorans).

For example, ethanol is produced at 2.5-fold higher efficiency when the cellulose degrading Clostridium is cultured, together with Zymomonas mobilis, in a medium containing cellulose than alone (Leschine and Canale-Parola, Current Microbiology, 11:129-136, 1984).

A microbe mixture may be provided as a solid form (e.g., lyophilizate) or in a liquid suspension. It may be added to a medium at the same time as, before or after Clostridium phytofermentans.

9. Butanol Production

Without a chemical treatment with an acid or base or a physical treatment of high pressure/high temperature, cellulosic biomass can be converted into butanol at high efficiency and yield by pure biological treatment and saccharification.

Production processes of biobutanol may be divided into a pre-treatment process, a saccharification process, a fermentation process and a purification process.

In butanol fermentation, optimized saccharification and fermentation processes can be simultaneously performed in a fermenter to produce glucose at a high yield. In the present invention, the cellulosic biomass is a non-lignocellulosic biomass derived from cellulose of microalgae.

Typically, a saccharification process is classified into an acidic saccharification process and a biological saccharification process. In acid saccharification, dil. or conc. acid is used to destroy cellulose and hemicelluloses structures to produce sugars. Enzyme saccharification, a kind of biological saccharification, recruits cellulase which degrades cellulose into cellobiose. This compound is a reducing disaccharide, with an empirical formula C₁₂H₂₂O₁₁, is colorless in a crystalline form, and is hydrolyzed into two molecules of glucose by β-glucosidase.

In an enzyme saccharification process, β-glucosidase immobilized on a support can be applied to the fraction of the microalgae cell culture solution to degrade the biomass of the cell culture solution, thereby producing glucose.

A non-enzyme biological saccharification method uses Clostridium thermocellum in saccharifying hemicelluloses and cellulose of microalgae (Biotechnology Letters Vol 7 No 7 509-514 (1985)).

Butanol fermentation may be carried out using an anaerobic microorganism selected from among, but not limited to, Clostridium acetobutylicum, Clostridium saccharoperbutylacetonicum, and Clostridium beijerinckii.

Isolation and purification of produced butanol from the cell culture solution can be achieved by pervaporation, extraction, distillation, gas stripping or adsorption. In addition, a hydrophobic ionic liquid may be employed to isolate butanol.

10. Organic Acid (Lactic Acid) Production

Lactic acid (2-hydroxypropanoic acid) may be produced by fermentation or chemically synthesized. Among lactic acid bacteria are Carnobacterium, Enterococcus, Lactobacillus, Lactococcus, Leuconostoc, Oenococcus, Pediococcus, Streptococcus, Tetragenococcus, Vagococcus and Weissella. In the present invention, a single strain (Lactobacillus brevis subsp. brevis) is used for anaerobic fermentation.

Lactic acid finds a broad spectrum of applications as a food additive and in other industries. Lactic acid is very important as an intermediate of biodegradable polymers, environmentally friendly solvents, plant growth regulators, and special chemicals. Petroleum-based synthetic lactic acid is advantageous in production cost, but is not suitable for use in the production of biodegradable polymers such as PLA (polylactate) because of the co-existence of D(−) and L(+) configurations. Great demand is expected for PLA because it is not only an alternative to the non-degradable plastics polyethylene, polystyrene, and polypropylene, all derived in the petroleum chemical industry, but is also biocompatible.

When lactic acid is produced through a fermentation process, a recovery process is needed to remove various impurities from the product. In addition, because the separation and purification process of all processes accounts for more than 50% of the total production cost of lactic acid, efficient recovery from the fermented liquid is indispensible for economical production of lactic acid. For the separation and purification of lactic acid, solvent extraction, electric dialysis, ion exchange chromatography, nanofiltration, or reverse osmosis may be used.

After an organic acid fermentation process to give a fermented liquid containing lactic acid and impurities, such as proteins, the microorganisms are removed by centrifugation, and lactic acid can be isolated from the fermented liquid by adding Ca(OH)₂ to form a precipitate of calcium lactate (Ca(LA)₂), and recovering the precipitate.

### MODE FOR INVENTION

A better understanding of the present invention may be obtained through the following examples which are set forth to illustrate, but are not to be construed as limiting the present invention.

### Example 1

### Hydrolysis/Acidogenic Fermentation of Organic Waste (Food Waste)

From food waste, matter that was difficult for microorganisms to degrade (chicken bones, fish bones, wood pieces) was removed. The food waste was finely ground to form a soup-like state using a grinder in order to facilitate subsequent processes including contact with microorganisms and transportation. The ground food waste was mixed at a ratio of 1:1 with water so that it could be sufficiently stirred, and was provided with oxygen before transfer to the fermenter 210.

In order to conduct hydrolysis and acidogenic fermentation in a semi-anaerobic condition, air was fed at the bottom and a middle part of the fermenter 210 using a compressor, and a stirrer mounted on the upper end of the fermenter was operated to allow the food waste to smoothly contact fermentation strains and oxygen.

The fermenter 210 was a 5 L bioreactor (Bioflo 3000) and the strains were thermophilic bacteria (Table 1). After the food waste was mixed at a ratio of 1:1 with water, the resulting 3 L mixture was introduced into the fermenter 210 and incubated at 50° C. for 24 hrs to kill putrefaction bacteria.

The fermenter was cooled to 45° C. and inoculated with 50 mL of the cultured strains, followed by incubation for 2˜5 days. The resulting fermented liquid (organic wastewater) was transferred to the organic wastewater accommodation device 230. The fermented liquid was found to have a TCOD of about 45,000 mg/L, and an SCOD of about 31,000 mg/L. A total content of nitrogen ranged from 3,600 to 4,800 mg/L with a mean value of 4,200 mg/L. A total content of phosphorus was measured at about 6.1 mg/L. Approximately 75% of the organic acids produced in the semi-anaerobic hydrolysis/acidogenic fermentation was accounted for by acetic acid.

### Example 2

### Cell Culture

Chlorella protothecoides which was maintained on a proteose agar slant was used in the present invention. One liter of a basic medium was prepared using KH₂PO₄ (0.7 g), K₂HPO₄ (0.3 g), MgSO₄.7H₂O (0.3 g), FeSO₄.7H₂O (3 mg). urea (1 g), Arnon's A5 solution (1 ml), and thiamine hydrochloride (10 μg), pH 6.3. Cell culturing was conducted at 20° C. under a 15,000 lux fluorescent lamp in a 5% CO2 atmosphere. Arnon's A5 solution comprised H₃BOS₃ (2.9 g), MnCl₂.4H₂O (1.8 g), ZnSO₄.7H₂O (0.22 g), CuSO₄.5H₂O (0.08 g), and MoO₃ (0.018 g) per liter. Heterotrophic fermentation of Chlorella protothecoides was conducted in the same basic medium containing 0.01% urea and 4.0% glucose, instead of 0.1% urea.

### Example 3

### Effect of Organic Wastewater on Microalgae (Cell)

The organic wastewater was diluted to lower the nitrogen concentration to 150 mg/l, inoculated with C. protothecoides, and incubated for 3 days. After 1 ml of the fermented C. protothecoides broth was diluted to 1/100,000, the dilution was spread over 1.5% agar plates, and the colonies thus formed were counted to evaluate the effect of the organic wastewater, that is, the fermented liquid of food wastes, on the growth of C. protothecoides. There were no significant differences in the growth of microalgae between incubation with the fermented food waste liquid (organic wastewater) and the basic medium.

The organic wastewater was diluted such that the dilution had a TCOD of 1,607 mg/l and a nitrogen concentration of 150 mg/l. Also, the dilution was adjusted to contain Mg²⁺ at a concentration of 500 mg/l and Ca²⁺ at a concentration of 150 mg/l, and phosphorus was added in such an amount that it was present at a ratio of 1:10 with nitrogen in the dilution.

### Example 4

### Nitrogen Removal by Microalgae Culture

C. protothecoides was grown to the log phase in the organic wastewater (fermented food waste liquid) in a 5 L culturing device 10 while stirring at 150 rpm. The microalgae were inoculated at a density of 1×10⁶ cells/ml, and a density of 5×10⁵˜1×10⁷ cells/ml was acceptable.

A quantitative examination of the removal of TCOD and nitrogen from the organic wastewater by microalgae fermentation was made. The fermented food waste liquid (organic wastewater: total nitrogen concentration 4,200 mg/l) was diluted such that the nitrogen concentration was 100, 150, 300 or 500 mg/l. Each dilution was added in an amount of 3,000 ml to the 5 L fermenter. In the dilutions, the TCOD of the organic wastewater (45,000 mg/l) was reduced to 1,071, 1,607, 3,214, and 5,357 mg/l, respectively.

After C. protothecoides was inoculated to the fermenter, the nitrogen concentration was monitored. At each nitrogen concentration, the nitrogen removal rate by C. protothecoides was analyzed. From the organic wastewater containing nitrogen at a concentration of 100, 150, 300, and 500 mg/l, nitrogen was removed at 38, 50, 33 and 21%, respectively, after culturing C. protothecoides for 4 days. A peak nitrogen removal rate was detected at a nitrogen concentration of 150 mg/l, with nitrogen remaining at a concentration of 75.6 mg/l.

### Example 5

### Effect of Fat-Soluble Material Extracting Solvent on Cell

A C. protothecoides culture was treated at a ratio 5:1 with a fat-soluble material extracting solvent of C10˜C16 alkanes for 5 min, followed by fractionation. A 1/100,000-fold dilution of 1 ml of the C. protothecoides fraction was spread over 1.5% agar plates, and the colonies thus formed were counted to evaluate the effect of the fat-soluble material extracting solvent on the growth of C. protothecoides. The results are given in FIG. 7. As shown in FIG. 7, the fat-soluble material extracting solvent had no influence on the growth of the cells.

### Example 6

### Cell Maturation and Fractionation of Fat-Soluble Material-Solvent

C. protothecoides was grown to the log phase in a 5 L culturing device 10 under a 15,000 lux light or in a dark condition while stirring at 150 rpm, and then concentrated to 500 g/L in a cell concentration device 300 equipped with a filtration-type concentrator. In a cell maturation device 400, the concentrated C. protothecoides was allowed to mature for 24 hrs in a maturation medium, free of limiting nutrients, containing 5% glucose.

### Example 7

### Effect of Fat-Soluble Material Extracting Solvent and Ultrasonic Vibrational Disintegration on Extraction of Intracellular Fat-Soluble Material

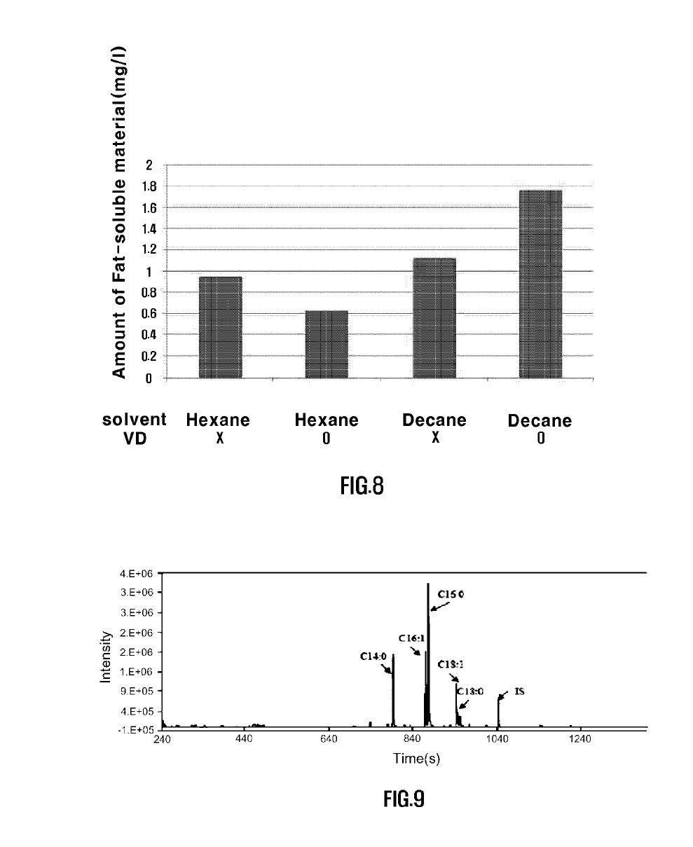

A culture of C. protothecoides grown to the log phase was treated for 5 min at a ratio of 5:1 with a fat-soluble material extracting solvent containing* hexane and decane, followed by vibrational disintegration (VD) at 40 kHz for 2 sec in a water bath. The fat-soluble material extracted with the fat-soluble material extracting solvent was saponified, and its free fatty acids were analyzed by LC-MC, with C17 used as a standard. The results are given in FIGS. 8 and 9. The treatment with the fat-soluble material extracting solvent for 5 min and vibrational disintegration for 2 sec extracted fatty acids. Upon the use of decane, the short-term vibrational disintegration increased the extraction efficiency of fat-soluble materials by 75%.

### Example 8

### Effect of Fat-Soluble Material Extracting Solvent and Ultrasonic Resonance Field on Extraction of Intracellular Fat-Soluble Material

A culture of C. protothecoides grown to the log phase was treated for 5 min at a ratio of 5:1 with a fat-soluble material extracting solvent containing hexane and decane, and transferred to a separation device 40 equipped with an acoustic cell filter 41 wherein the cells were separated. Then, the residue was delivered to a fractionation device 50 wherein a fat-soluble material-solvent was fractioned.

The acoustic cell filter 41, as shown in FIG. 2, comprised an acoustic chamber 42, a 3 MHz ultrasonic generator 43, an ultrasonic transducer 44, and a reflector 45. The acoustic chamber 42 was made of an acrylic tube, and the reflector 45 was glass. In the acoustic chamber 42, cells were observed to aggregate in the presence of an ultrasonic resonance field.

The fat-soluble material extracted with the fat-soluble material extracting solvent was saponified, and its free fatty acids were analyzed by LC-MC, with C17 used as a standard. The results are given in FIG. 9. The treatment with the fat-soluble material extracting solvent for 5 min and acoustic cell filtration extracted fatty acids.

### Example 9

### Isolation of Cells and Fractionation of Fat-Soluble Material-Solvent

C. protothecoides was grown to the log phase in a 5 L culturing device 10 under 15,000 lux light or in a dark condition while stirring at 150 rpm.

A cell culture of the culturing device 10 and a decane solvent (fat-soluble material extracting solvent) of the solvent device 20 were transferred at a ratio of 5:1 to the mixing device 30, and mixed.

The resulting mixture was delivered to the separation device 40 in which an acoustic cell filter as the ultrasonic resonance field generator 41, or a CS 10 Cell Settler (Biotechnology Solutions, USA) as the gravitational settling device 46 was operated. In the separation device 40, the cells formed aggregates, settling down while the remaining solution was continuously transferred to the fractionation device 50 where it was fractioned into an upper fat-soluble material-solvent layer and a lower water layer.

Subsequently, the cells separated in the separation device 40 were transferred via the cell circulation line 80 to the culturing device 10 where they were recultured in a cell medium. Meanwhile, the fat-soluble material-solvent (layer) fractioned in the fractionation device 50 was recycled via the solvent circulation line 90 to the solvent device 20.

### Example 10

### Re-Cultivation of Cells (High Density)

The cells, obtained in Examples 6 and 9, which were separated after extraction of fat-soluble materials were re-cultured. That is, the cell culture solution from which fat-soluble materials were extracted with the fat-soluble material extracting solvent was recycled to the culturing device 10, and re-cultured. They were found to grow at a rate of 0.028 g/h. When chlorella is grown to a high density, the growth inhibitor chlorellin is secreted therefrom, so that the cells stagnate at an almost fixed density. In this Example, when fat-soluble materials are extracted with the fat-soluble material extracting solvent, the secreted chlorellin was inferred to be removed, too, so that the cells could be cultured at a high density.

In consideration of a typical growth rate of about 0.033 g/h in a normal condition, the data obtained above indicate that the recycled cells normally grew without problems.

The culturing, mixing, separating and fractionating processes were carried out once a day, and optionally repeated 2 to 20 times at a frequency of one round a day to increase the density of cells to 50-200 gfw/L. From the concentrated cells, a fat-soluble material-solvent containing concentrated fat-soluble materials was obtained. The cell density and the concentration of fat-soluble materials were observed to increase by two to three times every round of repetition.

### Example 11

### Influence of Re-Maturation of Cell on Productivity

In order to evaluate the effect of re-maturation on cell productivity, biomass and fat-soluble materials were quantitatively analyzed in the C. protothecoides cells which were re-cultured and re-matured after the primary round of maturation and then the removal of fat-soluble materials, and in the C. protothecoides cells which were re-matured without re-culturing after the primary round of maturation and then the removal of fat-soluble materials.

The fat-soluble material-deprived C. protothecoides separated in the separation device 40 was delivered via the cell circulation line 80 to the culturing device 10 where they were re-cultured for 24 hrs with the cell culture solution. Then, the cells were concentrated to 500 g/L, and re-matured for 24 hrs, 48 hrs and 72 hrs, as in Example 2.

Separately, the fat-soluble material-deprived C. protothecoides cells separated in the separation device 40 in Example 2 were not re-cultured, but were transferred via the cell circulation line 80 to the cell maturation device 400 (FIG. 5) where they were re-matured for 24 hrs, 48 hrs and 72 hrs in a limiting nutrient-free maturation medium containing 5% glucose.

The resulting cell maturation solution in the cell maturation device 400 were delivered, together with a decane solvent (fat-soluble material extracting solvent) in the solvent device 20 to the mixing device 30, and mixed at a ratio of 1:1.

The resulting mixture was delivered to the separation device 40 in which an acoustic cell filter as the ultrasonic resonance field generator 41, or a CS 10 Cell settler (Biotechnology Solutions, USA) as the gravitational settling device 46 was operated. In the separation device 40, the cells formed aggregates, settling down while the remaining solution was continuously transferred to the fractionation device 50 where it was fractioned into an upper fat-soluble material-solvent layer and a lower water layer. The settled C. protothecoides was quantitatively analyzed for biomass

In this Example, the fat-soluble material-deprived C. protothecoides was inoculated at a concentration of 100 g/L, re-concentrated, re-measured at a density of 500 g/L in a maturation medium for 24 hrs, 48 hrs, and 72 hrs, re-separated, and re-fractionated. Biomass, fatty acids and carotenoid were measured in the cells after re-maturation for various times, and the results are summarized in Table 2, below.

TABLE 2

| Time | Biomass (g/L) | Fatty Acid | Carotenoid (g/L) |
|---|---|---|---|
| 0 hr | 126 | 25 | 0.01 |
| 24 hrs | 660 | 310 | 0.31 |
| 48 hrs | 675 | 360 | 0.35 |
| 72 hrs | 710 | 430 | 0.43 |

Note.
Data at zero hr detected in a cell culture inoculated at 100 g/L; data at 24, 48 and 72 hrs detected in matured solution inoculated at 500 g/L

In Table 2, effects of cell maturation time on the productivity of biomass and fat-soluble materials are shown after fat-soluble material-deprived Chlorella protothecoides cells underwent one round of the processes of reculturing, re-concentration, re-maturation, re-separation and re-fractionation.

Higher contents of biomass, and fat-soluble materials including fatty acids were detected when fat-soluble material-deprived C. protothecoides cells were inoculated at a density of 100 g/L, recultured, and rematured, than when fat-soluble material-deprived C. protothecoides cells were inoculated at a density of 100 g/L and re-cultured but not re-matured.

Again, the fat-soluble material-deprived C. protothecoides were inoculated at a concentration of 100 g/L and subjected to many rounds of the entire process of re-culturing for 24 hrs, re-concentration, re-maturation at a concentration of 500 g/L in a maturation medium for 24 hrs, re-separation, and re-fractionation. The production of biomass, fatty acids, and carotenoid according to the round of the entire processes is summarized in Table 3, below. Higher productivity was observed in round 1 than round 2 or 3.

TABLE 3

| | Biomass (g/L) | Fatty Acid | Carotenoid (g/L) |
|---|---|---|---|
| Round 1 | 660 | 310 | 0.31 |
| Round 2 | 675 | 215 | 0.23 |
| Round 3 | 680 | 225 | 0.23 |

Table 3 shows the effect of the number of repetition of the entire processes of re-culturing, re-concentration, re-maturation, re-separation and re-fractionation on the productivity of biomass and fat-soluble materials.

Separately, the fat-soluble material-deprived C. protothecoides cells were inoculated at a concentration of 500 g/L without re-culturing, re-matured for 24 hrs, 48 hrs or 72 hrs, re-separated, and re-fractionated. The production of biomass, fatty acids, and carotenoid according to re-maturation time is summarized in Table 4, below.

TABLE 4

| Time | Biomass (g/L) | Fatty Acid | Carotenoid (g/L) |
|---|---|---|---|
| 24 hrs | 660 | 310 | 0.31 |
| 48 hrs | 725 | 465 | 0.35 |
| 72 hrs | 765 | 515 | 0.55 |

Table 4 shows the effect of the re maturation time on the productivity of biomass and fat-soluble materials after one round of the entire processes of re-maturation without re-culturing, re-separation and re-fractionation was carried out.

When the fat-soluble material-deprived C. protothecoides cells were inoculated at a concentration of 500 g/L without re-culturing, re-matured, re-separated, and re-fractionation, the production of biomass, fatty acids, and carotenoid was increased in a re-maturation time-dependent manner.

Separately, the fat-soluble material-deprived C. protothecoides cells were inoculated at a concentration of 500 g/L, re-matured for 24 hrs without re-culturing, re-separated, and re-fractionated. Again, the cells were subjected to many rounds of the entire process. The production of biomass, fatty acids, and carotenoid according to the round of the entire processes is summarized in Table 5, below.

TABLE 5

| | Biomass (g/L) | Fatty Acid | Carotenoid (g/L) |
|---|---|---|---|
| Round 1 | 660 | 310 | 0.31 |
| Round 2 | 690 | 405 | 0.30 |
| Round 3 | 625 | 370 | 0.35 |

Table 5 shows the effect of the number of repetition of the entire processes of re-maturation without re-culturing, re-separation and re-fractionation on the productivity of biomass and fat-soluble materials.

There were no significant changes in the production of biomass, and fat-soluble materials including fatty acids with the number of repetition of the entire process after direct inoculation of C. protothecoides at a density of 500 g/L without re-culturing.

### Example 12

### Extraction of Fat-Soluble Material by Solvent Spraying

After C. protothecoides which was cultured as described in Examples 2 and 3, concentrated into 500 g/L and matured for 24 hrs was mixed at a ratio of 5:1 (v/v) with a decane solvent (fat-soluble material extracting solvent), the solvent was directly sprayed to the cell maturation solution so that it contacted the matured cells. The solvent was cycled for 4 hrs, and the fat-soluble material-solvent layer thus formed was removed and subjected to LC-MS. When fat-soluble materials were extracted in a continuous manner from the C. protothecoides which had been cultured, concentrated and matured, the fat-soluble materials and carotenoid were produced at a concentration of 305 g/L and 0.31 g/L, respectively.

### Example 13

### Extraction of Fatty Acid from Fat-Soluble Material-Solvent Fraction

The extraction of fatty acids, fat-soluble, was carried out using Buchi 210/215 Rotavapor (Buchi, Swiss) equipped with a round-bottom flask as the fat-soluble material solvent accommodation device 70. The fractionated fat-soluble material-solvent was transferred to the evaporator and placed in the round-bottom flask. Cold water was introduced into the condenser while the oil bath of the distillation flask was set at 174° C.

When distillation started, decane gas was allowed to flow into the condenser, and collected in a liquid phase in the accommodation flask. After the distillation was terminated, the volume of the decane collected in the accommodation flask, and the volume of the cell-derived fat-soluble materials remaining in the distillation flask were measured, and analyzed by LC-MS, with C17 serving as a standard. The recovered decane solvent was recycled to the solvent device 20.

### Example 14

### Extraction of Biodiesel from Cellular Fatty Acid

After being prepared by reacting methanol with sodium hydroxide, methoxide was reacted with the extracted cellular fat-soluble materials while stirring, to produce biodiesel, glycerin and a soap solid component. These products were centrifuged to divide biodiesel from glycerin and the soap component due to a difference in specific gravity.

The lower layer containing glycerin and the solid component was discharged into a separate glycerin storage tank while the upper layer of biodiesel was stirred together with two volumes of water so that glycerin, soap components and methanol present in the biodiesel were dissolved in the water. The resulting solution was centrifuged again to separate the biodiesel from the impurities including glycerin. The waste solution containing the impurities was discharged through a separate line to the glycerin storage tank. Finally, a distillation process was performed using an evaporator to evaporate water contained in an amount of 1˜2% in the biodiesel. The resulting biodiesel was analyzed by GC-TOF-MS (Gas Chromatography/Time of flight/Mass spectrometry; GC-6890N, Agilent Technologies, USA). The results is given in FIG. 9.

### Example 15

### Content of β-Carotene in Fat-Soluble Material-Solvent

The content of β-carotene in the fat-soluble material-solvent fraction was measured using HPLC (Hewlett Packard Series model 1100) equipped with Waters Spherisorb S5 ODS2 cartridge column (4.6×250 mm).

To isolate the pigment, the solvent was loaded at a flow rate of 1.0 ml/min in such a way that a solvent mixture of acetonitrile 90%, distilled water 9.99% and triethylamine 0.01% was flowed between 0 and 1 min, a solvent mixture of acetonitrile 86%, distilled water 8.99%, triethylamine 0.01%, and ethylacetate 5% between 2 and 14 min, and a solvent of 100% ethylacetate between 15 and 21 min. For a post-run analysis, the initial solvent was loaded for 9 min. When the reference wavelength (Jin et al., 2001. Biochim Biophys Acta 1506:244-2597) was 550 nm, the β-carotene pigment was detected at 445 nm. On the basis of a standard curve drawn from known amounts of β-carotene (DHI water and environment, Denmark), the content of β-carotene was determined to be 8.72×10-10 μM.

### Example 16

### Ethanol Fermentation of Fractionated Cells

As the fat-soluble material solvent accommodation device 70, a microbial fermenter (INNO 200603, InnoBio, Korea) was employed.

In a culture tube, Clostridium phytofermentans was grown in a GS-2 medium containing predetermined amounts of the fractionated cells.

In 1 liter of the GS-2 medium, 6 g of a yeast extract, 2.1 g of urea, 2.9 g of K₂HPO₄, 1.5 g of KH₂PO₄, 10.0 g of MOPS, 3.0 g of trisodium citrate dehydrate, and 2.0 g of cysteine hydrochloride were contained. Initially, the medium had a pH of 7.5 and contained Clostridium phytofermentans at a density of 0.8-1.1×10⁷ cells/mL. It was cultured while N₂ gas maintained at 30° C. was introduced.

At the time when the fermentation of the fractionated cells was completed, the concentration of ethanol was determined. Clostridium phytofermentans generated hydrogen immediately on ethanol fermentation. Ethanol was quantitatively analyzed by HPLC (Breeze HPLC system, Waters Co., USA) equipped with an RI detector. An Aminex HPX-87H column (3007.8 mm, Bio-rad) was employed.

Ethanol was measured to have a concentration of 0.23-0.26% (v/v) when the cells were used at a density of 10 g/L, and 0.42-0.54% (v/v) when the cells were used at a density of 20 g/L.

When the fractionated cells were used at a density of 40 g/L, the concentration of ethanol was measured to be 0.92-1.20% (v/v). Ethanol was distilled using a distillation column.

The results indicate that the fractionated cells, even though increasing in higher density, do not inhibit the action of Clostridium phytofermentans because the concentration of ethanol increased with the density of the fractionated cells. That is, Clostridium phytofermentans can ferment the cellular cellulose material into ethanol without pre-treating the fractionated cells with chemicals or cellulase or other enzymes.

### Example 17

### Extraction of Ethanol

The product of ethanol fermentation was transferred to a distillation column where ethanol distillation was conducted. In this regard, an oil bath was operated to heat the product to a temperature higher than the evaporating point of ethanol so as to evaporate ethanol. When the temperature was higher than the evaporating point of water, water evaporated and was mixed with ethanol. Thus, after the evaporating point was set at 78.3˜85° C., the product was heated for a certain period of time to evaporate ethanol which was condensed and stored in a separate storage tank.

### Example 18

### Butanol Fermentation of Fractionated Cells

As the fat-soluble material solvent accommodation device 70, a microbial fermenter (INNO 200603, InnoBio, Korea) was employed.

In a DSM medium, Clostridium thermocellum was anaerobically cultured at 60° C. while stirring at 150 rpm. The fractionated cells were delivered to a 5 L fermenter and inoculated with the C. thermocellum culture (5%, v/v), followed by saccharification by incubating at 60° C. for 3 days in an anaerobic condition while stirring at 150 rpm [Biotechnology Letters Vol 7 No 7 509˜514 (1985)]. The anaerobic condition was maintained by introducing nitrogen gas into the cell culture after inoculation.

For butanol fermentation, Clostridium acetobutylicum in a spore suspension was heated at 80° C. for 10 min, and then anaerobically cultured at 37° C. in a medium.

The fermenter was inoculated with the C. acetobutylicum culture (5%, v/v), followed by anaerobic butanol fermentation at 37° C. with stirring at 180 rpm. During fermentation, samples were taken periodically, and analyzed for butanol concentration.

In 1 liter of the DSM medium, 1.3 g of (NH₄)₂SO₄, 2.6 g of MgCl₂.6H₂O, 1.43 g of KH₂PO₄, 7.2 g of K₂HPO₄. 3H₂O, 0.13 g of CaCl₂.6H₂O, 1.1 mg of FeSO₄.7H₂O, 6.0 g of sodium β-glycerophosphate, 4.5 g of yeast extract, 10 g of a carbon source (filter paper, cellulosic mass, or cellobiose), 0.25 g of reduced glutathione and 1 mg of resazurin were contained. Its pH was adjusted into 5.0˜8.0 with 1 M HCl or 1 M NaOH.

The C. acetobutylicum culture medium contained 0.75 g of KH₂PO4, 0.75 g of K₂HPO₄, 0.4 g of MgSO₄.H₂O, 0.01 g of MnSO₄.H₂O, 0.01 g of FeSO₄. 7H₂O, 0.5 g of cysteine; 5 g of yeast extract, 2 g of asparagine-H₂O, 2 g of (NH₄)₂SO₄ per liter.

The products produced by the microorganisms after the sequential processes, that is acetone, butanol, and ethanol were quantitatively analyzed by gas chromatography (Agilent technology 6890N Network GC system) equipped with FID (Flame Ionization Detector). An HP-INNOWAX column (30 cm×250 μm×0.25 μm, Agilent technology) was employed. The temperature of both the sample injector and the detector was set to be 250° C. while the oven was heated from 50° C. to 170° C. at a rate of 10° C./min. The fermented liquid was found to contain 13 g/L butanol, 8 g/L acetone, and 0.5 g/L ethanol.

### Example 19

### Extraction of Butanol

The ionic liquid BMIM-TFSI imide [-butyl-3-methyl imidazolium bis(trifluoromethylsulfonyl)imide], and BMIM-PF6 (1-butyl-3-methyl imidazolium hexafluorophosphate) were used for butanol extraction. The fermented liquid in the fermenter was mixed at a ratio of 1:1 (v/v) with BMIM-TFSI (Sigma Aldrich, U.S.A.) or with BMIM-PF6 (Sigma Aldrich, U.S.A.). These mixtures were vortexed to extract butanol. An ionic liquid prepared using a typical method can be used. The amount of butanol was found to 60±1% upon extraction with BMIM-TFSI and 64±1% upon extraction with BMIM-PF6.

### Example 20

### Organic Acid Fermentation in Fractionated Cells

As the fat-soluble material solvent accommodation device 70, a microbial fermenter (INNO 200603, InnoBio, Korea) was employed.

The lactic acid bacterium Lactobacillus brevis subsp. brevis was grown in a PYG medium (peptone 20.0 g, glucose 5.0 g, yeast powder 10.0 g, NaCl 0.08 g, cysteine hydrochloride 0.5 g, calcium chloride 0.008 g, MgSO₄ 0.008 g, K₂HPO₄ 0.04 g, KH₂PO₄ 0.04 g, sodium bicarbonate 0.4 g, pH 7.1-7.3 per liter) in the fermenter, harvested by centrifugation, and washed with 0.085% saline. This bacterium was inoculated into the cells transferred to a reactor. Initially, the medium had a pH of 7.2, and contained the lactic acid strain at a density of 0.8˜1.1×10⁸ cells/mL. It was cultured while N₂ gas maintained at 30° C. was introduced. At the time when the fermentation of the fractionated cells was completed, the concentrations of the organic acids malate, lactate, acetate, citrate and butyrate were determined.

Concentrations of malate, lactate, acetate and citrate were measured by HPLC (HP placard, Japan) equipped with a Platinum EPS C18 organic acid analysis column (250 mm×4.6 mm, 5 μm) using 0.05M KH₂PO₄ at pH 2.4. Butyrate analysis was conducted by gas chromatography (HP placard, Japan) using a CP 58 Wax (FFAP) (30 cm×0.25 mm ID, 0.25 μm) column.

As a result, malate, lactate, acetate, citrate and butyrate were observed to have a concentration of 870.30±13.15, 746.16±8.91, 4,233.23±76.06, 318.04±47.75, and 1.99±1.99 mM, respectively.

The data indicates that the lactic acid stain can ferment a cellular cellulose material into lactic acid without pre-treating the fractionated cellular material with chemicals or cellulase or other enzymes.

### Example 21

### Extraction of Lactic Acid

The fermented liquid was adjusted into pH 10 with Ca(OH)₂, and heated in order to increase the solubility of the calcium lactate (Ca-lactate) thus formed, kill the bacteria, and solidify proteins, followed by filtration at a high temperature. The filtrate containing calcium lactate was cooled to precipitate calcium lactate. This precipitate was dissolved at a high temperature, and treated with sulfuric acid to form a CaSO₄ precipitate while recovering lactic acid as a solution.

1. A method for producing cells and fat-soluble materials by cell culture, comprising:
culturing cells containing fat-soluble materials;
contacting and mixing the cell culture solution with a fat-soluble material extracting solvent to exude the fat-soluble material into the fat-soluble material extracting solvent;
separating the cells from the mixed solution;
fractionating the remaining solution free of cells into a fat-soluble material-solvent layer containing the fat-soluble material, and a water layer.

2. The method of claim 1, further comprising concentrating cells of the cell culture solution.

3. The method of claim 1, further comprising maturing cells of the cell culture solution to allow the cells to have an increased content of the fat-soluble material, and modifying the cells such that the fat-soluble material extracting solvent readily penetrates into the cells to dissolve the fat-soluble material easily.

4. The method of claim 1, wherein the cells separated from the mixed solution are re-cultured, the resulting cell culture solution are mixed and contacted with the fat-soluble material extracting solvent or the fractionated fat-soluble material-solvent to further dissolve the fat-soluble material of the cells in the fat-soluble material extracting solvent, and the subsequent steps are repeated in such a way that a serial process of re-culturing the cells, re-dissolving the fat-soluble material, re-separating the cells, and re-fractionating the fat-soluble material-solvent is repeated 2 or more times, whereby the cells can be grown to a high density of interest, with concomitant production of the fat-soluble material-solvent containing a desired concentration of the fat-soluble material.

5. A method for producing cells and fat-soluble materials by cell culture, comprising:
culturing cells containing fat-soluble materials;
concentrating the cells of the cell culture solution;
maturing the cells to allow the cells to have an increased content of the fat-soluble material, and modifying the cells such that a fat-soluble material extracting solvent readily penetrates into the cells to dissolve the fat-soluble material easily;
contacting and mixing the concentrated and matured cell culture solution with the fat-soluble material extracting solvent to exude the fat-soluble material into the fat-soluble material extracting solvent;
repeating a series of processes of dividing a fat-soluble material-solvent as a separate layer in the fat-soluble material extracting solvent, said fat-soluble material-solvent containing the fat-soluble material, contacting the separate fat-soluble material-solvent with the matured cell culture solution to again divide the fat-soluble material-solvent as a separate layer in the fat-soluble material extracting solvent; and
obtaining the cells, and the fat-soluble material-solvent in which the fat-soluble material is dissolved, separately.

6. The method of claim 1, wherein while the cells are cultured, the cells ferment organic waste to produce organic wastewater containing low-molecular weight organic acids and biogas, said organic wastewater being recycled as nutrients in culturing the cells.

7. The method of claim 6, wherein the organic wastewater is diluted to an extent of a TCOD (total chemical oxygen demand) of 100 to 10,000 mg/l and a nitrogen concentration of 100 to 800 mg/l before being used in culturing the cells.

8. The method of claim 1, wherein the cells are cultured in organic wastewater containing an organic material, nitrogen and phosphorus, so that the organic material, nitrogen, and phosphorous are removed from the organic wastewater by using as nutrients of the cells, thereby purifying the organic wastewater.

9. The method of claim 1, wherein the fat-soluble material extracting solvent is a hydrocarbon solvent.

10. The method of claim 1, further comprising recovering a cell growth inhibitor and/or a pathogen from the cell culture solution, said cell growth inhibitor and said pathogen being secreted from the cells.

11. The method of claim 1, wherein the cells are separated from the mixed solution by applying a gravitational settling process or an ultrasonic resonance field to the mixed solution.

12. The method of claim 1, wherein the cell is mixed with the fat-soluble material extracting solvent through either or both of vibrational disintegration and stirring, whereby the cell culture solution is brought into improved contact with the fat-soluble material extracting solvent.

13. The method of claim 1, wherein the cells from which the fat-soluble material is extracted are used to produce at least one compound selected from among cellulose, hemicelluloses, monosaccharides, and oligosaccharides.

14. The method of claim 13, wherein hydrogen is generated and obtained when the compound is produced.

15. The method of claim 1, wherein the fractionated fat-soluble material-solvent is used as a source from which a fatty acid or a pigment is separated.

16. The method of claim 2, wherein the cells are concentrated by applying an ultrasonic resonance field or a gravitational settling method to the cell culture solution.

17. The method of claim 3, wherein the cell culture solution is supplemented with a carbon source absolutely or almost free of a limiting nutrient to establish a nutrient limiting condition which induces the cells to produce the fat-soluble material.

18. The method of claim 17, wherein the limiting nutrient is selected from the group consisting of a nitrogen source, a carbon source, a phosphate source, a vitamin source, a trace metal source, a major metal source, a silica source, and a combination thereof.

19. The method of claim 3, wherein the cell culture is controlled in dissolved oxygen content so as to induce the cells to mature.

20. An apparatus for producing cells and fat-soluble materials, comprising:
a culturing device 10 for culturing cells containing fat-soluble materials;
a solvent device 20 for storing and providing a fat-soluble material extracting solvent capable of dissolving the fat-soluble material in the cells;
a mixing device 30 for mixing the cell culture solution transferred from the culturing device 10 with the fat-soluble material extracting solvent transferred from the solvent device 20;
a separation device 40 for separating the cells from the mixed solution of the mixing device 30;
a fractionation device 50 for fractionating the cell-removed solution of the separation device 40 into a fat-soluble material-solvent layer comprising the fat-soluble material extracting solvent and the fat-soluble material, and a water layer;
a cell accommodation device 60 for accommodating or treating the cells separated in the separation device 40; and
a fat-soluble material solvent accommodation device 70 for accommodating or treating the fat-soluble material-solvent layer divided in the fractionation device 50.

21. The apparatus of claim 20, wherein the separation device 40 comprises an ultrasonic resonance field generator 41 for applying an ultrasonic resonance field to the mixed solution to separate the cells from the mixed solution, or a gravitational settling device 46 for applying gravitational settling to the mixed solution to separate the cells from the mixed solution.

22. The apparatus of claim 20, wherein the apparatus further comprises a cell circulation line 80 through which the cells separated in the separation device 40 is recycled to the culturing device 10; and a solvent circulation line 90 through which the fat-soluble material-solvent fractioned in the fractionation device 50 is recycled to the solvent device 20,
wherein the cell accommodation device 60 functions to accommodate or treat a high density of the cells finally separated in the separation device 40 after said cells are recycled once or more times through cell circulation line 80 to the culturing device 10 and re-cultured; and
the fat-soluble material-solvent accommodation device 70 functions to accommodate or treat the fat-soluble material-solvent finally fractionated in the fractionation device 50 after the fat-soluble material-solvent fraction is recycled once or more times through the solvent circulation line 90 to the fraction device 50 and re-fractionated.

23. The apparatus of claim 22, further comprising:
a first peristaltic pump 2 for feeding the cell culture solution of the culturing device 10 and the fat-soluble material extracting solvent of the solvent device 20 in a predetermine amount to the mixing device 30;
a second peristaltic pump 3 for selectively delivering the cells separated in the separation device 40 to the culturing device through the cell circulation line 80 or to the cell accommodation device 60 according to the density of the cells; and
a third peristaltic pump 4 for transferring the fat-soluble material-solvent fractionated in the fractionation device 50 to the solvent device 20 or the fat-soluble material solvent accommodation device 70 according to the concentration of the fat-soluble material-solvent.

24. The apparatus of claim 20, wherein the mixing device 30 further comprises a device selected from among a vibrational disintegrator 31, a stirrer 32, and a combination thereof, said vibrational disintegrator 31 functioning to disassemble aggregates of cells, said stirrer 32 functioning to the mixed solution, whereby the cell culture solution can brought into improved contact with the fat-soluble material extracting solvent.

25. The apparatus of claim 20, further comprising:
a fermenter 210 for fermenting organic waste to produce organic wastewater and biogas;
a biogas accommodation device 220 for capturing the biogas; and
an organic wastewater accommodation device 230 for storing the organic wastewater, wherein the organic wastewater fed from the organic wastewater accommodation device 230 is transferred to the culturing device 10 and used in culturing the cells.

26. An apparatus for producing cells and fat-soluble materials, comprising:
a culturing device 10 for culturing cells containing fat-soluble materials;
a solvent device 20 for storing and providing a fat-soluble material extracting solvent capable of dissolving the fat-soluble material of the cells;
a cell concentration device 300 for concentrating cells of the cell culture solution from the culturing device 10;
a cell maturation device 400 for maturing the concentrated cells of the cell culture solution;
a mixing device 30 for mixing the cell culture solution transferred from the culturing device 10 with the fat-soluble material extracting solvent transferred from the solvent device 20;
a separation device 40 for separating the cells from the mixed solution of the mixing device 30;
a fractionation device 50 for fractionating the cell-removed solution of the separation device 40 into a fat-soluble material-solvent layer comprising the fat-soluble material extracting solvent and the fat-soluble material, and a water layer;
a cell accommodation device 60 for accommodating or treating the cells separated in the separation device 40;
a fat-soluble material solvent accommodation device 70 for accommodating or treating the fat-soluble material-solvent layer divided in the fractionation device 50; and
a water accommodation device 240 for accommodating or treating the water separated in the cell concentration device 300 and the fractionation device 50.

27. The apparatus of claim 26, wherein the cell maturation device 400, the mixing device 30, and the separation device 40 are assembled into an integrated separation device 500.

28. The apparatus of claim 27, wherein the integrated separation device 500 is designed to allow the fat-soluble material extracting solvent to be fed to a lower portion of the cell culture solution, and comprises an inlet line 510 through which the fat-soluble material extracting solvent is introduced from the solvent device 20, a spout tube 520, connected to the end of the inlet line 510, for spouting the fat-soluble material extracting solvent upward from the bottom of the cell culture solution, a recovery line 530 through the fat-soluble material extracting solvent of the integrated separation device 500 plus the fat-soluble material dissolved in the fat-soluble material extracting solvent are recovered to the solvent device 20, and a peristaltic pump 540, located on both the inlet line 510 and the recovery line 530, for functioning to provide a pressure necessary for transferring the solvents.

29. The apparatus of claim 28, wherein the integrated separation device 500 further comprises a stirrer 550 for mixing the cell culture solution with the fat-soluble material extracting solvent.

30. The apparatus of claim 26, wherein the cell concentration device 300, the cell maturation device 400, the mixing device 30, and the separation device 40 are integrated into a single device.
